(19) United States
(12) Patent Application Publication
(10) Pub. No.: US 2015/0297619 A1
(43) Pub. Date: Oct. 22, 2015

(54) METHODS AND COMPOSITIONS FOR PRESERVING THE MUCOSAL BARRIER

(71) Applicant: The Regents of the University of California, Oakland, CA (US)

(72) Inventors: Geert SCHMID-SCHONBEIN, Del Mar, CA (US); Angelina ALTSHULER, Danville, CA (US); Alexander PENN, San Diego, CA (US)

(21) Appl. No.: 14/674,897

(22) Filed: Mar. 31, 2015

Related U.S. Application Data
(60) Provisional application No. 61/980,737, filed on Apr. 17, 2014.

(51) Int. Cl. A61K 31/7004; A61K 31/196; A61K 31/65

(52) U.S. Cl. CPC A61K 31/7004; A61K 31/65; A61K 31/196

(57) ABSTRACT

Provided herein are methods and compositions for preventing small molecule permeability of the small intestine curing total ischemia by administering glucose to the lumen of the intestine, as well as administration of serine protease or metalloproteinase inhibitors. Also provided are compositions for performing the same.

### CROSS REFERENCE TO RELATED APPLICATION(S)

This application claims the benefit of priority under 35 U.S.C. §119(e) of U.S. Ser. No. 61/980,737, filed Apr. 17, 2014, the entire content of which is incorporated herein by reference.

### GRANT INFORMATION

This invention was made with government support under Grant No. GM-85072 awarded by the National Institutes of Health. The United States government has certain rights in the invention.

### BACKGROUND OF THE INVENTION

1. Field of the Invention

The invention relates generally to enteral glucose administration to preserve the mucosal barrier and more specifically to treatments for reducing or inhibiting larger molecule weight permeability increase of the intestinal mucosa (for, e.g., pancreatic digestive enzymes).

2. Background Information

Intestinal ischemia is an important problem in critical care that can be caused by trauma or sepsis and is accompanied by an increase in small intestine permeability as measured by transport from the intestinal lumen into the wall of the intestine and into the blood. Under normal conditions, the mucin and epithelium with its tight junctions compose the mucosal epithelial barrier that is responsible for compartmentalizing the intestinal contents, including pancreatic digestive enzymes and digesting food particles, in the lumen. During ischemia, epithelial opening of tight junctions, cell shedding and loss of the attached mucin cause this barrier to fail. The reduced perfusion to the intestine results in damage to the intestinal villi and other components of the intestinal wall. The permeability increases and, as a result, intestinal contents may leak across the mucosal barrier. After escape from the intestinal lumen, intestinal contents can be transported through the venous intestinal vasculature, lymphatics, or after passage across the intestinal wall via the peritoneum into the systemic circulation, and may be responsible for distant organ injury. Therefore, there is a need for methods and compositions for preventing breakdown of the barrier to reduce organ injury and improve survival following shock.

### SUMMARY OF THE INVENTION

In intestinal ischemia, inflammatory mediators in the small intestine's lumen such as food breakdown products, bacteria, viruses and digestive enzymes leak into the peritoneal space, lymph, and circulation, but the mechanisms by which the intestinal wall permeability initially increases at the time shock occurs are not well defined. It is therefore hypothesized that wall protease activity (independent of luminal proteases) of an acutely ischemic small intestine and apoptosis contribute to the increased transmural permeability of the intestine's wall. To model intestinal ischemia, the proximal jejunum to the distal ileum in the rat was excised, the lumen was rapidly flushed with saline to remove luminal contents, sectioned into equal length segments, and filled with a tracer (fluorescein) in saline, glucose, or protease inhibitors. The transmural fluorescein transport was determined over 2 hours. Villi structure and epithelial junctional proteins were analyzed. After ischemia, there was increased transmural permeability, loss of villi structure, and destruction of epithelial proteins. Supplementation with luminal glucose preserved the epithelium and significantly attenuated permeability and villi damage. Matrix metalloproteinase (MMP) inhibitors (doxycycline, GM-6001), and serine protease inhibitor (tranexamic acid) in the lumen, significantly reduced the fluorescein transport compared to saline for 90 min of ischemia. Based on these results, in vivo model of hemorrhagic shock (HS, 90 min 30 mmHg, 3 hours observation) was used to test for intestinal lesion formation. Single enteral interventions (saline, glucose, tranexamic acid) did not prevent intestinal lesions, while the combination of enteral glucose and tranexamic acid prevented lesion formation after hemorrhagic shock. The results suggest that apoptotic (by reduction of ATP values due to, e.g., reduced oxygen supply) and protease mediated breakdown cause increased permeability and damage to the intestinal wall. Metabolic support in the lumen of an ischemic intestine with glucose reduces the transport from the lumen across the wall and enteral proteolytic inhibition attenuates tissue breakdown. These combined interventions ameliorate lesion formation in the small intestine after hemorrhagic shock.

The methods provided herein encompass both metabolic support to the lumen of the intestine to prevent failure of the mucosal barrier and protease inhibition to prevent the intestine from autodigestion by proteases in the tissue itself and by proteases in the lumen of the intestine. Additionally, the invention further provides for administration of protease inhibitors to the central circulation to block any active digestive enzymes that may escape from the lumen or become active due to the initial injury (ischemia, trauma, burns, etc.). These strategies may be used in all forms of hypotension, trauma, sepsis, necrotizing enterocolitis, or surgeries where the intestine will be ischemic for extended periods of time (e.g., after open heart surgery or heart transplant) or where epithelial cells may be stimulated to increase mucosal permeability.

Accordingly, in one aspect, the present invention provides a method for preventing and/or minimizing breakdown of a mucosal barrier in the intestine of a subject in need thereof. The method includes administering to the lumen of the intestine an effective amount of glucose. The glucose may be administered to the subject orally, by nasogastric intubation (NG tube), or by catheter into the duodenum. The method may further include administering an effective dose of a matrix-degrading metalloproteinase (MMP) inhibitor to the subject. The MMP inhibitor may be administered orally, and may be selected from the group consisting of doxycycline, minocycline, minocycline analogs, tetracyclin-based inhibitors, hydroxamate-, thiol-, phosphorus-, pyrimidine-based inhibitors, iliomastat, tranexamic acid, endogenous tissue inhibitors of metalloproteinase (TIMPs), grape seed extract, resveratrol, and GM-6001. The method may further include administering an effective dose of a serine protease inhibitor, such as t tranexamic acid, 6-amidino-2-naphthyl p-guanidinobenzoate dimethanesulfonate nafamostat mesilate (ANGD), gabaxate mesilate (Foy), trasylol, alpha 1-antitrypsin, kallikrein, neutrophil elastase inhibitor, plasminogen activator inhibitor-1, and alpha 1-antichymotrypsin (α₁AC), to the subject. The subject may be a mammal, such as a human.

In another aspect, the invention provides a method for treating sepsis in a subject in need thereof. The method includes administering to the lumen of the intestine of the subject an effective amount of glucose. The subject may be suffering from intestinal complications associated with hypoxia, trauma, hypothermia, burn, or a condition associated with intestinal underperfusion. In certain embodiments, the condition associated with intestinal underperfusion is selected from the group consisting of elevated central venous blood pressure, mesentery artery occlusion, and aortic occlusion. The glucose may be administered to the subject orally, by nasogastric intubation (NG tube), or by catheter into the duodenum. The method may further include administering an effective dose of a matrix-degrading metalloproteinase (MMP) inhibitor to the subject. The MMP inhibitor may be administered orally, and may be selected from the group consisting of doxycycline, minocycline, minocycline analogs, tetracyclin-based inhibitors, hydroxamate-, thiol-, phosphorus-, pyrimidine-based inhibitors, iliomastat, tranexamic acid, endogenous tissue inhibitors of metalloproteinase (TIMPs), grape seed extract, resveratrol, and GM-6001. The method may further include administering an effective dose of a serine protease inhibitor, such as tranexamic acid, 6-amidino-2-naphthyl p-guanidinobenzoate dimethanesulfonate nafamostat mesilate (ANGD), gabaxate mesilate (Foy), trasylol, alpha 1-antitrypsin, kallikrein, neutrophil elastase inhibitor, plasminogen activator inhibitor-1, and alpha 1-antichymotrypsin (α₁AC), to the subject. In certain embodiments, a combination of glucose, serine protease inhibitor, and/or MMP inhibitor may be co-administered to the subject. The subject may be a mammal, such as a human.

In another aspect, the invention provides a method for preventing and/or counteracting intestinal ischemia of the epithelial barrier in a trauma patient. The method includes administering an effective dose of glucose into the lumen of the intestine of the subject and an effective dose of a MMP inhibitor. The method includes administering to the lumen of the intestine of the subject an effective amount of glucose. The subject may be suffering from intestinal complications associated with hypoxia, trauma, hypothermia, burn, or a condition associated with intestinal underperfusion. In certain embodiments, the condition associated with intestinal underperfusion is selected from the group consisting of elevated central venous blood pressure, mesentery or splanchnic artery occlusion, and aortic occlusion. The glucose may be administered to the subject orally, by nasogastric intubation (NG tube), or by catheter into the duodenum. The MMP inhibitor may be administered orally, and may be selected from the group consisting of doxycycline, minocycline, minocycline analogs, tetracyclin-based inhibitors, hydroxamate-, thiol-, phosphorus-, pyrimidine-based inhibitors, iliomastat, tranexamic acid, endogenous tissue inhibitors of metalloproteinase (TIMPs), grape seed extract, resveratrol, and GM-6001. The method may further include administering an effective dose of a serine protease inhibitor, such as tranexamic acid, 6-amidino-2-naphthyl p-guanidinobenzoate dimethanesulfonate nafamostat mesilate (ANGD), gabaxate mesilate (Foy), trasylol, alpha 1-antitrypsin, kallikrein, neutrophil elastase inhibitor, plasminogen activator inhibitor-1, and alpha 1-antichymotrypsin (α₁AC), to the subject. In certain embodiments, a combination of glucose, serine protease inhibitor, and/or MMP inhibitor may be co-administered to the subject. The subject may be a mammal, such as a human.

In another aspect, the invention provides a method for preventing and/or counteracting intestinal ischemia of the epithelial barrier in a trauma patient. The method includes administering an effective dose of glucose into the lumen of the intestine of the subject and an effective dose of a serine protease inhibitor. The subject may be suffering from intestinal complications associated with hypoxia, trauma, hypothermia, burn, or a condition associated with intestinal underperfusion. In certain embodiments, the condition associated with intestinal underperfusion is selected from the group consisting of elevated central venous blood pressure, mesentery artery occlusion, and aortic occlusion. The glucose may be administered to the subject orally, by nasogastric intubation (NG tube), or by catheter into the duodenum. The serine protease inhibitor may be tranexamic acid, 6-amidino-2-naphthyl p-guanidinobenzoate dimethanesulfonate nafamostat mesilate (ANGD), gabaxate mesilate (Foy), trasylol, alpha 1-antitrypsin, kallikrein, neutrophil elastase inhibitor, plasminogen activator inhibitor-1, and alpha 1-antichymotrypsin (α₁AC). The method may further include administering an effective dose of a MMP inhibitor. The MMP inhibitor may be administered orally, and may be selected from the group consisting of doxycycline, minocycline, minocycline analogs, tetracyclin-based inhibitors, hydroxamate-, thiol-, phosphorus-, pyrimidine-based inhibitors, iliomastat, tranexamic acid, endogenous tissue inhibitors of metalloproteinase (TIMPs), grape seed extract, resveratrol, and GM-6001. In certain embodiments, a combination of glucose, serine protease inhibitor, and/or MMP inhibitor may be co-administered to the subject. The subject may be a mammal, such as a human.

### BRIEF DESCRIPTION OF THE DRAWINGS

FIGS. 1A-1D are graphical and pictorial diagrams showing fluorescein transport and tissue degradation in saline and glucose treated intestines after severe ischemia. The intestinal lumen was flushed of all food contents and digestive enzymes. (FIG. 1A) Fluorescent tracer transport rates across the wall of ischemic intestinal segments located along the length of the small intestine from the proximal jejunum (position 1) to the distal ileum (position 8). The fluorescein transport rates were computed as amount (nM) of fluorescein accumulating outside of the intestinal wall over 30 min time intervals. The intestinal segments were enterally filled with fluorescein in saline without and with glucose before ischemia began. p<0.05 by 2-way ANOVA, with Tukey post hoc test significance compared to Position 2 marked by ++ (during time interval T2), * (T3), ** (T4). p<0.05 by Tukey post hoc test compared to T1 shown by † (T2), ‡ (T3) and § (T4). (FIG. 1B) Intestinal wall morphology in the jejunum (top) and ileum (bottom) as seen on frozen sections after Van Gieson and hematoxylin labeling. Black arrows indicate intact villi structure that best matched the structure of the pre-ischemic tissue, white arrow shows separation between the lamina propria and the mucosal epithelial layer, (*) indicates sites of damaged villi, and (§) specifies internal damage to the villi. (FIG. 1C) TUNEL labeling of pre-ischemic control, saline, and glucose intestine segment and the tips. Black arrows point to intact villi. Brown stained nuclei indicate TUNEL-positive cells (brown arrows) while blue labeled nuclei indicate negative labeling (blue arrows). Positive and negative controls depict brown and blue stained nuclei, respectively. * indicates TUNEL positive cells in the muscularis. (FIG. 1D) Immunoblots of epithelial bound mucin 13, occludin, and E-cadherin. *, p<0.05 vs. pre-ischemic control tissues and ‡ compared to saline tissues by one way ANOVA with Tukey post hoc. N=6 rats/group for saline and N=5 rats/group for glucose. Mean±SEM.

FIGS. 2A-2B are graphical and pictorial diagrams showing protease activity in intestinal homogenates. (FIG. 2A) Gelatin gel zymographies showing protease activity in the pre-ischemic jejunum (segment 2) and ileum (segment 7), with and without renaturation in tranexamic acid. (FIG. 2B) Quantification of bands by densitometry. *, p<0.01 by paired t-test between jejunum vs. ileum. §, p<0.01 by paired t-test between No Inhibition and Tranexamic Acid (20 mM) renaturing. N=4/group. Mean±SEM.

FIGS. 3A-3D are graphical and pictorial diagrams showing MMP inhibition and intestinal wall destruction during ischemia. (FIG. 3A) Fluorescein transport across the wall of ischemic intestinal segments filled with tranexamic acid, or MMP inhibitors (doxycycline, GM 6001). p<0.05 by two way ANOVA with Tukey post hoc test significance compared to Position 2 shown by ** (during T4). Significant changes (p<0.05 by Tukey post hoc test) compared to T1 shown by ‡ (T3) and § (T4). N=3 rats/group. (FIG. 3B) Third order polynomial fit to measured mean fluorescein rates at each position for either the 30-60 min or 60-90 min period for tranexamic acid, doxycycline, GM 6001 and saline groups to compare fluorescein transport at 30-60 and 60-90 min of severe ischemia. Adjusted R2 values are 0.87, 0.37, 0.81, and 0.71 for 30-60 min; 0.80, 0.89, 0.97, and 0.86 for 60-90 min for saline, tranexamic acid, doxycycline and GM 6001 curves, respectively. Comparison of curve fits for tranexamic acid, doxycycline or GM 6001 with those for saline by F-test, p=5.6×10−4, 1.3×10−4 and 9.1×10−5 for 30-60 min; p=6.5×10−3, 1.8×10−3 and 4.3×10−3 for 60-90 min. (FIG. 3C) Representative images of the intestinal villi. Arrows indicate intact villi structure and (*) indicates sites of damaged villi after ischemia. (FIG. 3D) Immunoblots of epithelial bound mucin 13, occludin, and E-cadherin. *p<0.05 vs. pre-ischemic control tissues and ‡ compared to saline-ischemic tissues by one way ANOVA with Tukey post hoc. N=6 rats/group for pre-ischemic controls and tranexamic acid; N=3 rats/group for doxycycline and GM-6001. Mean±SEM.

FIGS. 4A-4C are graphical and pictorial diagrams showing the results from glucose or tranexamic acid intervention in hemorrhagic shock. (FIG. 4A) Gross morphology of the intestine in the rats before and after hemorrhagic shock. Lesions due to escape of red cells form in the HS+Saline animals and HS+Glucose treated animals (see magnified views) while the HS+Tranexamic Acid animals intestinal injury was in part reduced. (FIG. 4B) MPO activity measured in intestinal segments from segment 2, the region with the appearance of the most severe lesions, was elevated in all groups after hemorrhagic shock. “•” indicate individual data points for each animal. (FIG. 4C) Mean arterial pressure (MAP) of animals during the course of hemorrhagic shock. Data are presented as mean±SD. N=6 rats/group for No-HS and HS+Saline; N=7 rats/group for HS+Glucose and HS+Tranexamic Acid. Scale bar equals 5 mm. Mean±SD.

FIGS. 5A-5C are graphical and pictorial diagrams showing the results from tranexamic acid or GM 6001 combined with glucose fluorescein transport. (FIG. 5A) Fluorescent tracer rates across the wall of ischemic intestinal segments filled with tranexamic acid+glucose or GM 6001+glucose in saline. N=3 rats/group. (FIG. 5B) Representative micrographs of intestinal villi with tranexamic acid+glucose or GM 6001+glucose after ischemia. Black arrows indicate intact villi structure similar to the pre-ischemic control (FIG. 1) and white arrows indicate points of separation between the lamina propria and the mucosal epithelial layer. (FIG. 5C) Separation between the lamina propria and the mucosal epithelial layer. *, p<0.0001 vs. pre-ischemic intestinal tissue. Refer to FIG. 1B for images of pre-ischemic and glucose treated intestines. ‡, p<0.0001 for glucose vs. glucose+tranexamic acid. N=3 rats/group. Mean±SEM.

FIGS. 6A-6C are graphical and pictorial diagrams showing the results from Hemorrhagic shock (HS) with enteral tranexamic acid+glucose. (FIG. 6A) Gross morphology of the intestine in rats before and after hemorrhagic shock. Lesions form in the HS+Saline animals but were reduced in the HS+Tranexamic Acid+Glucose treated animals (see magnified views). (FIG. 6B) MPO activity measured in intestinal segments from segment 2 trended to decrease in the HS+Tranexamic Acid+Glucose animals. “•” indicate individual data points for each animal and ‘x’ indicates an outlier. Bar graph shows mean±SD; outlier is excluded from the bar graph mean value in the HS+Tranexamic Acid+Glucose group. (FIG. 6C) Mean arterial pressure (MAP) of animals during the course of hemorrhagic shock. The MAP during the reperfusion period followed a linear trend for the HS+Saline animals. N=6 rats/group for HS+Saline; N=4 rats/group for HS+Tranexamic Acid+Glucose. Scale bar equals 5 mm. Mean±SD.

FIGS. 7A-7D are graphical and pictorial diagrams showing the results of intestinal injury with single treatments. (FIG. 7A) MAP records for the five groups. (FIG. 7B) Gross morphology of the intestine before and after HS. Length bar=0.5 cm. (FIG. 7C) Microhemmorhage formation into the intestinal tissue as measured by 405 nm absorbance and (FIG. 7D) MPO activity of intestinal wall homogenates. *, p<0.05 by ANOVA followed by Tukey post-hoc analysis. N=7 rats/group except for Sham where N=5 rats/group. Box and whisker plots showing median (central line), upper lines of the box corresponding to the first and third quartiles, respectively, and outliers.

FIGS. 8A-8D are graphical and pictorial diagrams showing serine protease transport and activity in the plasma. (FIG. 8A) Serine protease activity (˜20 kDa) as detected by gelatin gel zymography and (FIG. 8B) trypsin and chymotrypsin protein levels after HS in saline, doxycycline (DOX), tranexamic acid (TA) and glucose (GLUC) treated groups. (FIG. 8C) MMP-9 activity and neutrophil derived pro-MMP-9 activity. (FIG. 8D) MMP-9 and TIMP-1 protein levels before and after HS. *, p<0.05 by paired t-test. **, p<0.05 comparing post-HS+SAL to post-HS+GLUC by ANOVA followed by Tukey post hoc analysis. N=5 rats/group. Bar graphs show mean±SD.

FIGS. 9A-9C are graphical and pictorial diagrams showing lung injury after HS. (FIG. 9A) MPO activity of lung homogenates after HS. BALF protein concentration and MPO activity in the BALF increased with worsening condition in the lung, but there was high variability in these measurements. N=7 rats/group except for Sham where N=5 rats/group. (FIG. 9B) Serine protease activity bands after HS and trypsin protein levels in the lung after HS. (FIG. 9C) MMP-9 activity and protein levels in the lung. *, p<0.05 by comparison of post-HS samples by ANOVA followed by Tukey post-hoc test. N=4 rats/group. Bar graphs show mean±SD.

FIGS. 10A-10C are graphical and pictorial diagrams showing lung membrane protein degradation. (FIG. 10A) VE-cadherin was significantly reduced for HS+SAL, HS+DOX, and HS+TA treatments, but E-cadherin levels were not statistically different. Measurements are expressed as relative units (RU) protein levels normalized by No-HS controls. (FIG. 10B) VEGFR-2 protein levels in the lung. (FIG. 10C) Plasma levels of 38 kDa VEGF before and after shock. *, p<0.05 by comparison to No-HS samples and **, p<0.05 by comparison of HS+SAL and HS+TA by ANOVA followed by Tukey post-hoc test. §, p<0.01 by paired t-test. N=5 rats/group.

FIGS. 11A-11C are graphical and pictorial diagrams showing the results from combination treatments. (FIG. 11A) Gross morphology of the intestine before and after HS. Length bar=0.5 cm. (FIG. 11B) MAP of HS+SAL and HS+DOX+TA+GLUC. (FIG. 11C) Protein degradation in the lung for multiple treatments. *, p<0.05 by ANOVA with Tukey post-hoc correction. The MAP decreased at a significantly faster linear rate (p<0.01 by t-test) in the HS+SAL (−0.32±0.12 mmHg/min) animals compared to the HS+DOX+TA+GLUC (−0.04±0.12 mmHg/min). N=4 rats/group except No-HS and HS+SAL where N=7 rats/group.

### DETAILED DESCRIPTION OF THE INVENTION

The present invention is based on the finding that metabolic support to the lumen of the intestine to prevent failure of the mucosal barrier and protease inhibition to prevent the intestine from autodigestion by proteases in the tissue itself and by proteases in the lumen of the intestine. Additionally, the invention further provides for administration of protease inhibitors to the central circulation to block any active digestive enzymes that may escape from the lumen or become active due to the initial injury (ischemia, trauma, etc.).

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The term “subject” as used herein refers to any individual or patient to which the subject methods are performed. Generally the subject is human, although as will be appreciated by those in the art, the subject may be an animal. In various embodiments, the subject may be any animal having an intestine. Thus other animals, including mammals such as rodents (including mice, rats, hamsters and guinea pigs), cats, dogs, rabbits, farm animals including cows, horses, goats, sheep, pigs, and including sea mammals, etc., and primates (including monkeys, chimpanzees, orangutans and gorillas) are included within the definition of the term “subject”.

A “therapeutic effect,” as used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “administration” or “administering” are defined to include the act of providing a compound or pharmaceutical composition of the invention to a subject in need of treatment. The phrases “parenteral administration” and “administered parenterally” as used herein means modes of administration other than enteral and topical administration, usually by injection, and includes, without limitation, intravitreal, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection, intracameral and intravitreal injection, and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration. In various embodiments, the methods include, but are not limited to, administration to the subject orally, by nasogastric intubation (NG tube), by catheter into the duodenum or upper jejunum, or by direct administration to the small intestine. In certain embodiments, the methods include direct administration via a catheter that is passed through the abdominal wall (e.g., a feeding tube).

The term “co-administer” and “co-administering” and variants thereof refer to the simultaneous presence of two or more active agents in a subject. The active agents that are co-administered can be concurrently or sequentially delivered.

The digestive track has long been associated with the progression of shock and multiple organ dysfunction syndrome (MODS) in intensive care units (Lillehei 1957; Robinson et al. 1966), and the small intestine in particular is a major contributor to the dysfunction (Lillehei 1957; Poggetti et al. 1992; Magnotti et al. 1998). Hypovolemia from hemorrhage results in intestinal ischemia and subsequent apoptosis of epithelial cells (Lu et al. 2008). This results in epithelial shedding and loss of the attached mucin (Ikeda et al. 1998; Grossmann et al. 2002; Sheng et al. 2011; Chang et al. 2012a,b). Together the mucin and epithelial tight junctions form the mucosal barrier that is responsible for keeping the intestinal contents, including pancreatic digestive enzymes and digested food particles, compartmentalized in the intestine's lumen. Failure of the barrier as a result of ischemia allows the contents of the intestine to penetrate into the wall of the intestine and contribute to further intestinal damage (Chang et al. 2012a,b).

The presence of food in the intestine is a major factor that influences the outcome of shock conditions (Bounous 1967; Bounous et al. 1967). The luminal contents in the small intestine consist of digestive enzymes, food particles, bile, and some bacteria (far less than in the large intestine). They may be nonuniformly distributed along its length depending on the time of food ingestion and vary between trauma patients. Luminal contents may serve as proinflammatory mediators after the onset of intestinal ischemia contributing to the MODS that occurs during shock if they escape the intestine via the mesenteric lymph, circulation, or via transmural permeation into the peritoneal space where they may initiate inflammation in peripheral organs, for example, the lung (Ishimaru et al. 2004; Deitch 2010; Altshuler et al. 2012).

However, the benefits from the nutrient component of the luminal contents, for example, sugars and amino acids, help to maintain the mucosal epithelial barrier during intestinal ischemia (Chiu et al. 1970b; McArdle et al. 1972; Robinson and Mirkovitch 1977; Flynn et al. 1992), and thus may be of advantage to the intestine during shock. The main drawback of digesting food is that it is dissolved in a mixture of powerful degrading enzymes in the lumen of the intestine that may cross the mucosal epithelial barrier during the ischemic state (Chang et al. 2012b) when the epithelial layer becomes permeable (Ikeda et al. 1998; Grossmann et al. 2002). Inhibition of pancreatic enzymes in the lumen of the intestine reduces intestinal microhemorrhages, decreases distant organ injury, and improves survival after experimental shock (Mitsuoka and Schmid-Schonbein 2000; Mitsuoka et al. 2000; Deitch et al. 2003; Doucet et al. 2004; Shi et al. 2004; DeLano et al. 2013), suggesting a prime role for these enzymes in shock pathogenesis. However, the inhibitors may not only act on the enzymes within the intestinal lumen. If the inhibitors themselves cross the mucosal barrier, they may also affect enzymes in the intestinal wall, vasculature, and/or other organs. An alternative method for determining the contribution of the luminal content to hemorrhagic shock (HS) is needed, for example, flushing of the entire luminal content in a segment of the intestine that has previously been the target of intraluminal protease inhibitor injections prior to hypotension.

In a severe ischemic state, there may be multiple mechanisms for breakdown of the intestine, e.g., by depletion of ATP, including cell apoptotic processes, and proteolytic degradation. It has previously been shown that enteral treatment with protease inhibitors is protective during shock, but since low molecular weight inhibitors such as tranexamic acid may also be transported into the wall of the intestine, determining their mechanism of action is confounded by the presence of both pancreatic-derived digestive proteases in the intestinal lumen and proteases inherent to the intestinal tissue, and even bacterial proteases. Tranexamic acid is a synthetic analog of the amino acid lysine. It is typically used to treat or prevent excessive blood loss during surgery and in various other medical conditions. It is an antifibrinolytic that competitively inhibits the activation of plasminogen to plasmin, by binding to specific sites of both plasminogen and plasmin, a molecule responsible for the degradation of fibrin, a protein that forms the framework of blood clots.

Several potential sources of proteases in the intestinal tissue could be activated during ischemia and may contribute to the breakdown of the intestinal wall. One of the most prevalent classes of protease in the epithelial cells and the wall of the intestine are the matrix metalloproteinases (MMPs), capable of digesting the extracellular matrix. Endothelial cells in microvessels, and extravasated leukocytes are also potential sources of MMPs. If activated or released during ischemia, these enzymes could degrade the intestinal wall, enabling leakage of pro-inflammatory mediators derived from the lumen (proteases, bacteria, digested food particles, etc.) of the intestine into the peritoneum.

The objective of this study is to investigate the breakdown of the wall of the small intestine during ischemia by mechanisms inherent to the tissue, i.e., in the absence of luminal contents, and determine which degrading processes (cell death or protease degradation) contribute to transmural permeability of a low molecular weight tracer. It is hypothesized that in a model of severe intestinal ischemia metabolic support (e.g., glucose, which can be directly metabolized by enterocytes to ATP and has reduced epithelial shedding into the lumen during intestinal ischemia) helps to preserve the epithelial barrier resulting in minimal penetration of a low molecular weight tracer while protease inhibition in the lumen of the intestine prevents intestinal wall tissue breakdown.

Thus, an ex vivo approach was used, in which luminal content of an excised intestine is replaced with a low molecular weight tracer in saline with glucose as metabolic support and/or protease inhibitors that were recently tested as enteral interventions in shock models. Transmural permeability, morphological damage, the level of protease activity in the tissue, and junctional protein integrity were determined. This approach was chosen over in vivo models of total ischemia such as splanchnic arterial occlusion and ligation of vessels along isolated intestinal loops for two reasons: 1) complete ischemia is not assured by splanchnic arterial occlusion and 2) because of anatomical constraints, neither in vivo approach allows permeability to be measured as a function of distance along the intestine in the same animal. The present invention thus demonstrates the change in transmural permeability after total ischemia along the entire length of the jejunum and ileum. The results provided herein indicate that metabolic support to the epithelium preserves the mucosal barrier while enteral protease inhibition using tranexamic acid prevents structural breakdown of the wall. It is also shown that in hemorrhagic shock, individual treatments are not sufficient to protect the intestine to prevent intestinal hemorrhage and a decline blood pressure after reperfusion. However, enteral tranexamic acid plus glucose was effective at reducing transmural permeability and intestinal breakdown and prevented visible hemorrhage and stabilized blood pressure during reperfusion following hemorrhagic shock. Understanding the breakdown process of the intestinal wall may help in the design and implementation of new interventions against the escape of luminal contents into the peritoneum in conditions of intestinal ischemia.

Accordingly, in one aspect, the present invention provides a method for preventing breakdown of a mucosal barrier in the intestine of a subject in need thereof. The method includes administering to the lumen of the intestine an effective amount of glucose. The glucose may be administered to the subject orally, by nasogastric intubation (NG tube), or by catheter into the duodenum. As discussed below, the method may further include administering an effective dose of a matrix-degrading metalloproteinase (MMP) inhibitor to the subject. The MMP inhibitor may be administered orally, and may be any compound that inactivates MMPs. Exemplary MMP inhibitors useful in the methods of the present invention are selected from the group consisting of doxycycline, minocycline, minocycline analogs, tetracyclin-based inhibitors, hydroxamate-, thiol-, phosphorus-, pyrimidine-based inhibitors, iliomastat, tranexamic acid, endogenous tissue inhibitors of metalloproteinase (TIMPs), and natural compounds such as, grape seed extract, resveratrol, and others. (See, e.g., Jialiang, Philippe E. Van den Steen, Qing-Xiang A. Sang, Ghislain Opdenakker: Matrix metalloproteinase inhibitors as therapy for inflammatory and vascular diseases Nature Reviews Drug Discovery 6, 480-498, June 2007). In certain embodiments, the MMP inhibitor is GM-6001.

In another aspect, the study was designed to investigate early injury mechanisms in the intestine and lung following trauma/hemorrhagic shock and to examine whether proteolytic or metabolic intervention in the intestinal lumen prevents organ injury. First, IV and IP inhibition of MMPs using doxycycline will be used to block the MMPs activated in the intestine, circulation, and other organs during HS because their inhibition reduces vascular injury following ischemia/reperfusion. Second, enteral delivery of a serine protease inhibitor will be given, which has been shown to preserve the mucin layer and increase survival. Exemplary serine protease inhibitors useful in the methods of the invention include, but are not limited to tranexamic acid, 6-amidino-2-naphthyl p-guanidinobenzoate dimethanesulfonate nafamostat mesilate (ANGD), gabaxate mesilate (Foy), trasylol and serpins (e.g., alpha 1-antitrypsin, alpha 1-antichymotrypsins (α₁AC), kallikrein, neutrophil elastase inhibitor, and plasminogen activator inhibitor-1).

Enteral glucose as metabolic support for enterocytes has been effective in preventing breakdown of the mucosal epithelial barrier, but has remained relatively unexplored in models of hemorrhagic shock as an enteral resuscitation therapy. We hypothesize that if the gut barrier is preserved by protease inhibition or metabolic support to the intestine, intestine and lung injury will subside, and to a greater degree if the interventions are administered in combination. The results suggest that combined proteolytic inhibition in the periphery and intestine with metabolic support optimally reduce organ injury after HS.

As such, the invention also provides a method for treating sepsis in a subject in need thereof. The method includes administering to the lumen of the intestine of the subject an effective amount of glucose. The subject may be suffering from intestinal complications associated with various medical conditions. Exemplary medical conditions include, but are not limited to, hypoxia, trauma, anesthesia, radiation and/or chemotherapy, hypothermia, burn, systemic or intestinal infections (bacterial, viral), drug/alcohol overdose, lung injury, and any condition associated with intestinal underperfusion. In certain embodiments, the condition associated with intestinal underperfusion is selected from the group consisting of elevated central venous blood pressure, mesentery artery occlusion, and aortic occlusion.

In HS, intestinal injury at the macroscopic and molecular levels is more severe if luminal contents are present prior to the onset of HS (HS-NF group). At lesion sites, red cell escape and neutrophil accumulation positively correlate with proteolytic activity in intestinal homogenates of the HS-NF animals. However, the presence of luminal contents in the jejunum and ileum during HS affects neither the levels of serine proteases in the plasma, nor lung injury as measured by neutrophil infiltration and protein leakage. The protease activity increases in the mesenteric lymph fluid after intestinal ischemia, and the level of trypsin activity is increased in the lung in animals with a nonflushed intestine. Lung endothelial and epithelial proteins were degraded to a similar extent without and with the intestinal flush even though HS-NF animals had greater lung serine protease and MMP-9 activity and greater plasma total MMP-9 levels. The MAP of animals did not correlate with the degree of intestinal injury in the animals, indicating that at an early stage of fluid resuscitation degrading mechanisms may not necessarily be reflected in the blood pressure.

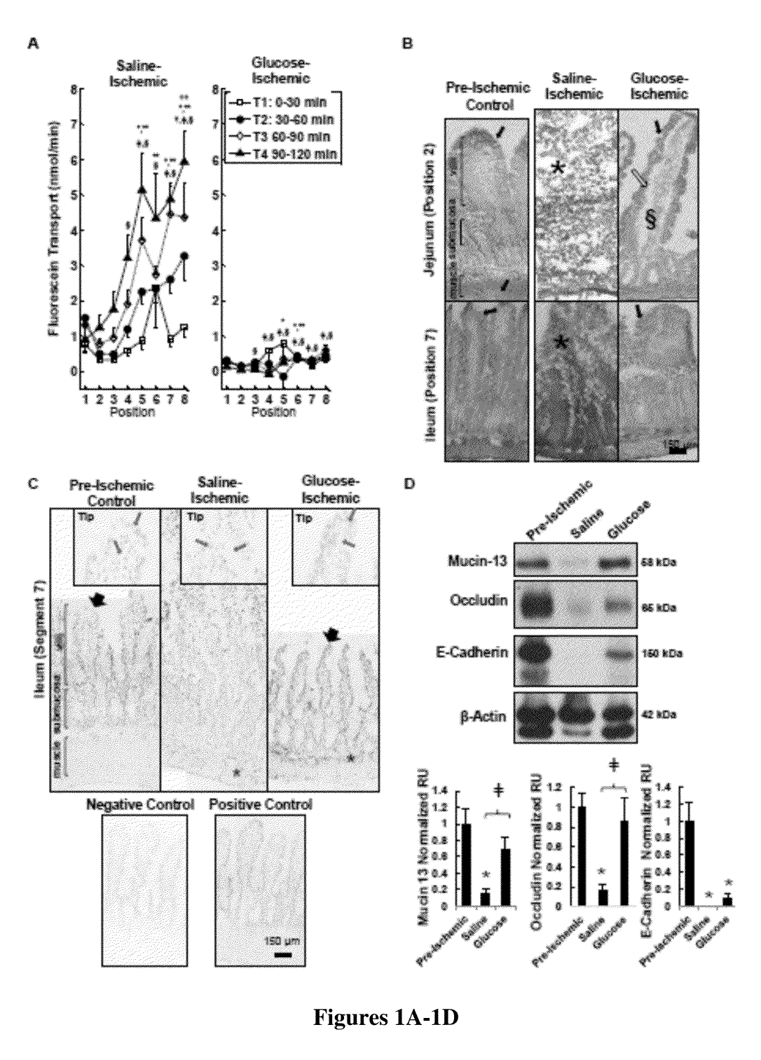

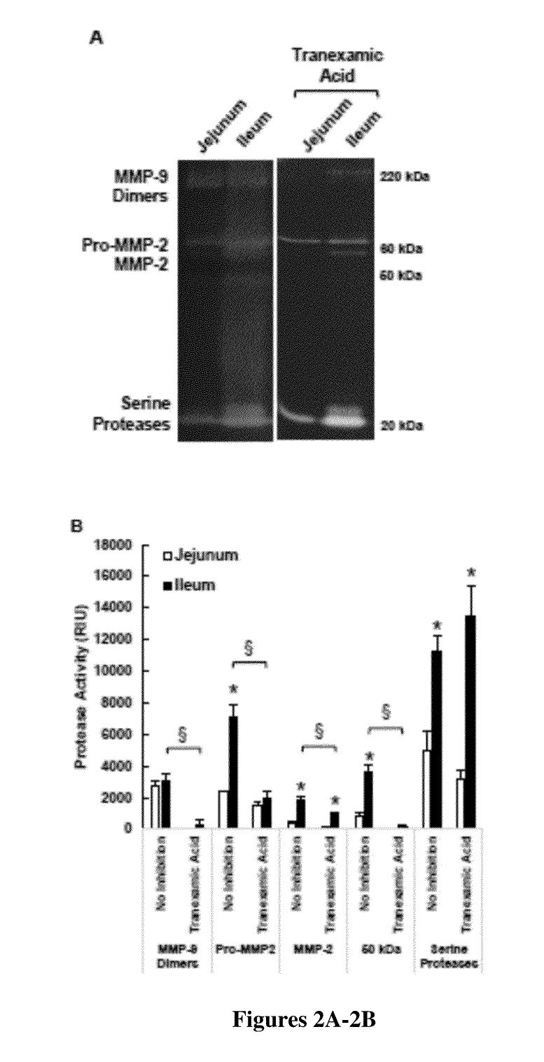

The results provided herein indicate that the transmural transport of a low molecular weight tracer from the lumen across the rat intestinal wall was undetectable in a non-ischemic state but increases after complete intestinal ischemia, even in the absence of its luminal contents. The permeability increased consistently from the jejunum to the ileum in all cases and may be a result of the increased basal protease activity in the ileal tissue (FIG. 2). Placement of glucose into the lumen of the small intestine abrogated the transmural permeability increase of a low molecular weight tracer, reduced apoptosis and loss of the tight junction protein occludin, and served to maintain the epithelial layer but not necessarily the underlying intestinal wall (FIG. 1B). In the absence of glucose, the MMP inhibitors doxycycline and GM 6001 and the protease inhibitor tranexamic acid reduced the transmural permeability through the 60-90 min time interval, but they did not prevent the villi destruction of the epithelium at 2 hrs. In acute hemorrhagic shock, unflushed intestines enterally pre-treated with tranexamic acid+glucose had reduced formation of macroscopic lesions and stabilized blood pressure compared to animals with enteral saline only. The evidence suggests that both the epithelium and the tissue under the epithelium act as barriers to the transmural passage of a low molecular weight tracer, and preventing both avenues of damage is necessary to reduce the intestinal injury after hemorrhagic shock.

Glucose preserves the epithelial barrier by preventing epithelial cell shedding—Glucose administration curtailed the rise in transmural permeability to fluorescein in the ischemic small intestine, likely by maintaining the epithelial barrier, in agreement with previous results showing preservation of the barrier properties during ischemia in the presence of luminal glucose. Despite blood flow cessation, glucose provides epithelial cells a direct source of metabolic energy to produce ATP, which has been shown to maintain the barrier in vitro. ATP supply, which could be achieved by glucose supplementation, is required for epithelial cells to maintain attachments and prevent excessive apoptosis and shedding of apoptotic epithelial cells into the lumen (anoikis) The presence of glucose in the lumen during ischemia prevented the cells from undergoing apoptosis (FIG. 1C). Elevated levels of apoptosis and epithelial shedding into the lumen can occur within 15 minutes of intestinal ischemia, which illustrates the sensitivity to ischemic conditions.

Partial protection with MMP inhibition—The intestine is rich in MMPs, and gelatinase activities were detected at approximately 50 kDa (MMP-1 or MMP-3), 60 kDa (MMP-2), and 220 kDa (MMP-9 dimer) similar to previous findings. Without being bound by theory, MMPs could originate from a variety of cells in the intestine, including immune cells such as neutrophils and mast cells in the intestine. Doxycycline and GM-6001 are both broad-spectrum MMP inhibitors (GM 6001 also inhibits ADAMs). Unexpectedly, tranexamic acid was also able to directly inhibit MMPs (FIG. 2A) in addition to serine proteases (e.g., trypsin). All three inhibitors reduced but did not prevent the increase in transmural permeability through the first 90 minutes of ischemia. By 120 minutes, permeability was no longer significantly lower than that in the saline group. It is currently unclear how much of this early preservation was due to protection of the intestinal extracellular matrix versus reduced destruction of the epithelial layer (FIG. 2B), since both the extracellular matrix and epithelial layer were damaged after 120 min of ischemia ex vivo. However, given that protection of the epithelial layer by glucose completely prevented permeability increase, it is likely that the epithelial barrier is the dominant barrier that controls transmural permeability and that partial prevention of permeability increase with MMP inhibition is due instead to effects on the extracellular matrix. Protection of extracellular matrix proteins from degradation may minimize the creation of pores through the lamina propria, muscularis, and serosa through which fluorescein could reach the outer compartment, even if the epithelial barrier were no longer intact.

In support of this idea, the tight junctional protein occludin was decreased by ischemia and preserved by glucose, but not doxycycline or GM-6001. Interestingly, E-cadherin, which was also decreased by ischemia, was not preserved with glucose, suggesting that epithelial barrier properties are maintained by other tight junctions rather than by adherence junctions only. Occludin and E-cadherin can be degraded by MMPs, however MMP inhibition did not curtail occludin or E-cadherin destruction, implying MMPs are not responsible for their reduction following ischemia. Alternatively, as the epithelium becomes apoptotic, occludin, E-cadherin, and mucin 13 may be internalized and subsequently degraded, possibly aided by lactic acid build up and reduced intracellular pH during ischemia. It should be noted that E-cadherin and mucin 13 also degrade in the presence of luminal proteases, and therefore their breakdown could be accelerated in cases of severe ischemia when luminal content is also present.

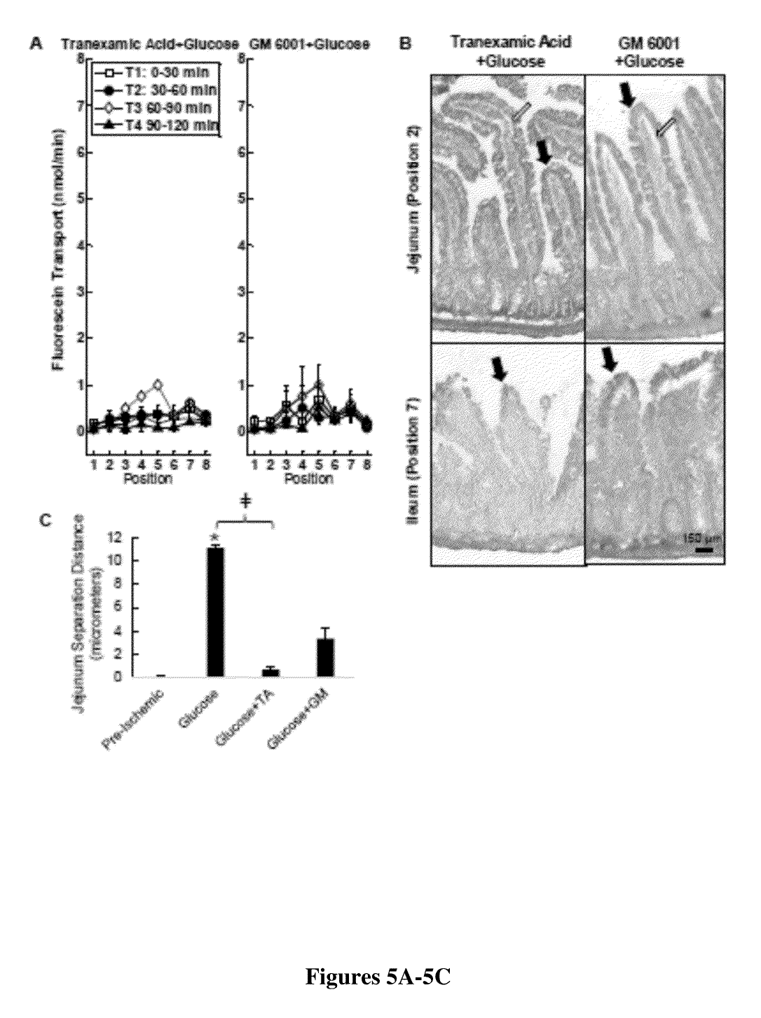

Tranexamic acid had additional protective effects that were not present with the other MMP inhibitors. It preserved mucin 13, which may improve epithelial survival in non-flushed intestines. The decrease in occludin levels, while not significantly better than saline animals, was also not significantly different from pre-ischemic levels in the presence of tranexamic acid. Histological results from the group with combined treatment of tranexamic acid and glucose suggests that tranexamic acid may also help the preservation of epithelial attachments to the basement membrane in the ex vivo study (FIG. 5C), a process which may reduce anoikis and preserve the epithelial barrier. Reduced anoikis may explain the improvements in mucin 13 and occludin. Without being bound by theory, the additional benefits over doxycycline and GM-6001 may be due to the fact that tranexamic acid, as a lysine analog, inhibits plasmin and conversion of plasminogen to plasmin. Plasmin is a trypsin-like enzyme that is a potent activator of MMPs and ADAMs (“a disintegrin and metalloproteinase”) expressed on epithelial cells and activated during ischemia. Inhibiting plasmin may also attenuate the consequent downstream activation of metallo- or other proteases that contribute to tissue destruction during ischemia

Jejunum and ileum have different permeability profiles—In all cases, the transmural permeability for fluorescein increased from the jejunum to the ileum (with a non-significant increased permeability in position 1 compared to position 2 along the length of the small intestine). The major transition from an impermeable to a permeable state occurred after 1 hour in positions 5 and higher, corresponding to the ileum. The ileum, often described as the most permeable region of the intestine, is associated with the most damage after trauma and/or shock and is where the majority of microhemorrhages in experimental shock animals and necrotizing enterocolitis occur. It was surprising that the morphology of the untreated ischemic jejunum appeared as disrupted as that of the untreated ischemic ileum, despite its lower transmural permeability. Since the epithelial layer was destroyed in both cases, which serves as the primary barrier to fluorescein, the differences between the jejunum and ileum lie beneath the epithelium.

When the proteolytic activity was measured by gelatin gel zymography, the ileum had a higher density of serine proteases and MMPs compared to the jejunum in pre-ischemic tissue (FIG. 2). These proteases could degrade the extracellular matrix structure of the villi, muscularis, and/or serosa more rapidly in the ileum than in the jejunum allowing fluorescein to pass with less resistance. Given that the difference between ileum and jejunum was primarily in magnitude of activity rather than composition of proteases, it is likely that if the ischemic period were extended, the jejunum would reach the same high levels of permeability as the ileum. The enhanced presence of proteases in the ileum could cleave integrin attachments between cells and the extracellular matrix, which may enhance apoptosis in the wall of the ileum at a greater rate than the jejunum. Detachment of cells from the extracellular matrix would provide an alternative route for fluorescein to penetrate through the intestinal tissue. Although the transmural permeability of the jejunum is lower than the ileum, it would not necessarily prevent egress through the blood or lymph in vivo.

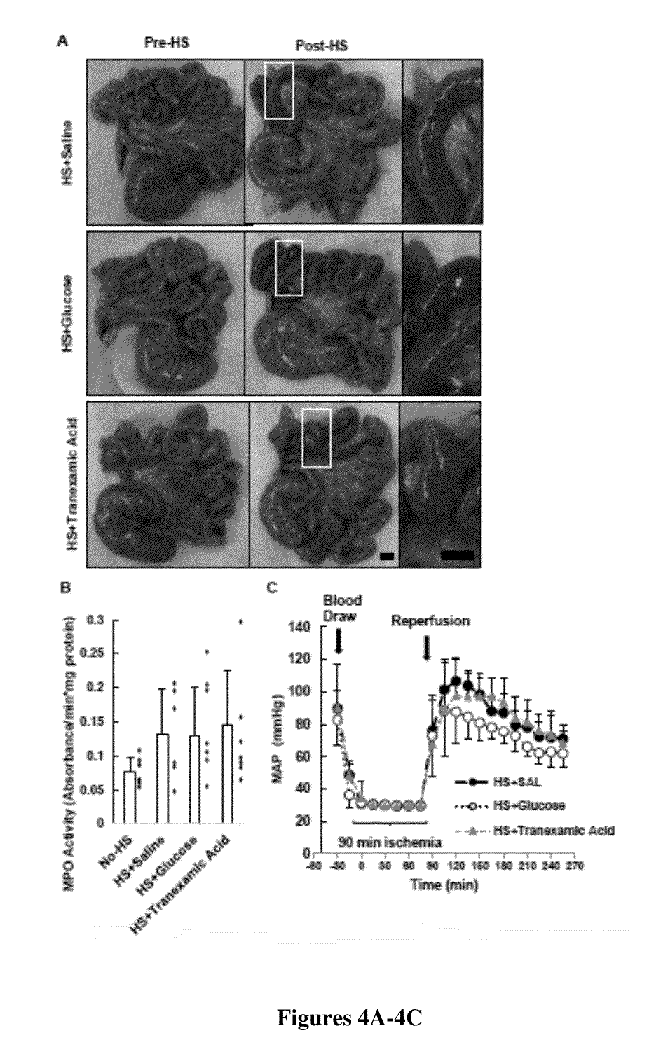

Application of tranexamic acid+glucose—The ex vivo model described herein was designed with a flushed intestine with no reperfusion. For the in vivo study, it was intended to evaluate the treatment groups in the harsher environment created by normal luminal content. As such, the efficacy of saline, glucose, tranexamic or the combination of the two was tested in the lumen of the intestine as a pretreatment for hemorrhagic shock in an unflushed intestine. Single interventions of either glucose or tranexamic acid did not prevent microhemorrhages from forming in the jejunum, decrease the neutrophil accumulation in the intestinal homogenates, or stabilize the MAP compared to saline alone (FIG. 4) which suggest that even though individual components of the gut may be preserved, the gut reperfusion injury can still occur.

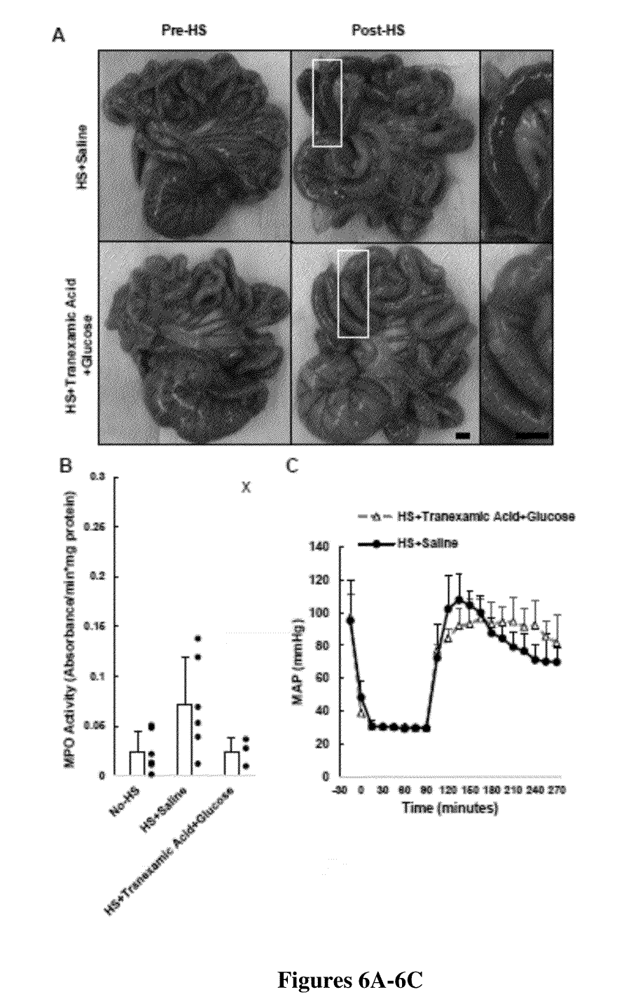

While combined tranexamic acid and glucose treatment improved the overall gross morphology of the intestine (FIG. 6A), lowered neutrophil infiltration in three of the four animals (FIG. 6B), and stabilized blood pressure (FIG. 6C), the presence of an outlier suggests there are other factors in the intestine that can attract neutrophils following ischemia/reperfusion injury. One such factor could be the presence of food, since these animals were eating ad libitum prior to the experiment. It has previously been shown that a greater generation of pro-inflammatory mediators in the gut may occur in regions containing partially digested food. Therefore the contents of the intestine may be important in the treatment and diagnosis of patients entering the intensive care unit after traumatic situations. Although tranexamic acid+glucose did reduce intestinal damage, future work needs to determine what components in the lumen attract neutrophils and how these components cause inflammation even in the presence of interventions that reduce transmural permeability and intestinal breakdown.

The transition from reversible to irreversible shock may be related to the severity of the epithelial barrier destruction and the extent to which full physiological gut barrier/digestive features can be restored. Therefore, patients undergoing elective surgery that anticipate intestinal ischemia may benefit from intestinal protection with intraluminal glucose supplementation in combination with tranexamic acid treatment in order to reduce intestinal damage.

Thus, in another aspect, the invention provides a method for preventing intestinal ischemia in a trauma patient. The method includes administering an effective dose of glucose into the lumen of the intestine of the subject and an effective dose of a MMP inhibitor. Likewise, the invention provides a method of for preventing intestinal ischemia in a trauma patient by administering an effective dose of glucose into the lumen of the intestine of the subject and an effective dose of a serine protease inhibitor.

Animals treated with a combination of interventions targeting proteolytic breakdown and metabolic support reduced organ injury following hemorrhagic shock. Intestinal gross morphology was improved by HS+TA, HS+DOX, and the multi-target interventions (apart from HS+DOX+GLUC). However, serine protease activity bands still formed in the lung homogenate and plasma samples. Pancreatic trypsin was detected after HS in the plasma and lung regardless of the intestine's condition. While protease inhibition with doxycycline reduced the plasma MMP activity by plate zymography and MMP-9 levels in the lung, it was unable to prevent protein degradation in the lung. Enteral tranexamic acid treatment effectively prevented VEGFR-2 degradation in the lung while circulating VEGF was elevated after HS in all groups. The multi-target intervention was able to stabilize the blood pressure, protect the intestine, and reduce protein degradation.

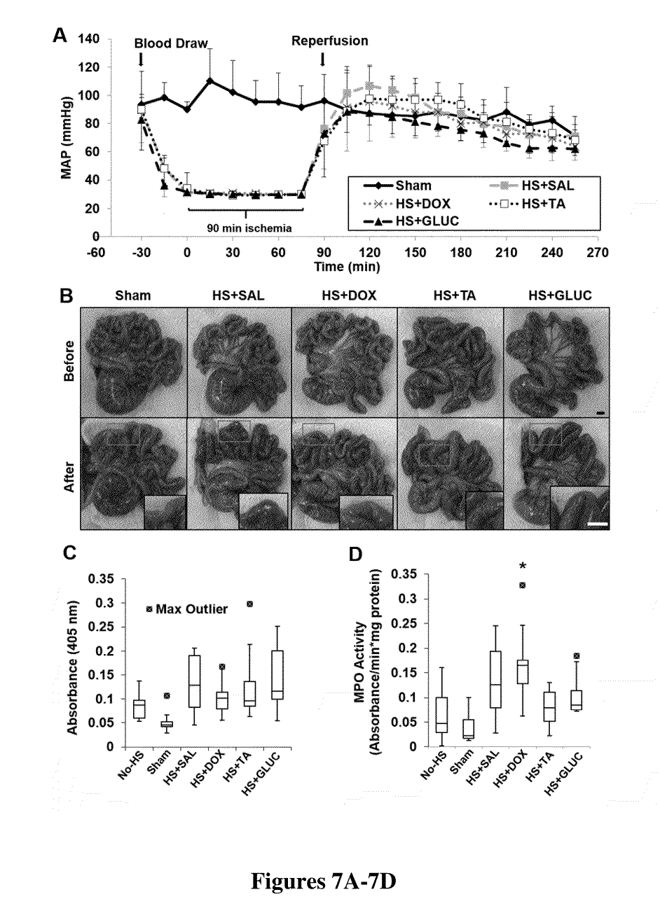

Intestinal Damage—Microhemorrhages, as observed by gross morphology, were common in the jejunum following HS, and were improved with intravenous doxycycline and enteral tranexamic acid treatment, suggesting that microhemorrhages are a proteolytically driven process (FIG. 7A). Glucose treated animals had visible hemorrhage into the intestine, which may be due to an increase in blood flow to the intestine in the presence of glucose and may explain why the combined HS+DOX+GLUC treatment still yielded microhemorrhages despite the presence of doxycycline.

It was observed that enhanced neutrophil presence in the intestine after HS, as measured by MPO activity. During the low flow state, neutrophils accumulate in the microcirculation, and as a result may become trapped and migrate into the intestinal tissue. MPO activity tended to be less in the HS+TA or HS+GLUC groups, potentially because they preserve the mucosal epithelial barrier and prevented luminal contents from penetrating into the intestinal wall, thus reducing the inflammatory signal, and/or because of improved microvascular blood pressure.

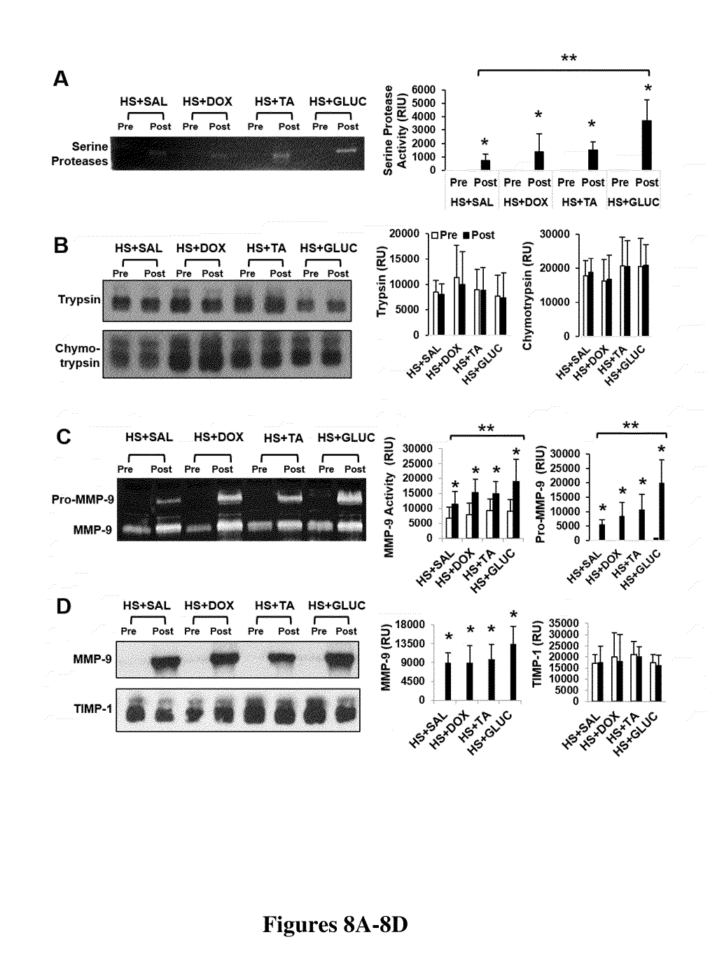

The animals that received glucose had elevated MMP-9 activity in the plasma (FIG. 8C), which suggests that the pharmacological concentration of glucose that was administered to protect the mucosal epithelial barrier may have caused more cells (e.g. neutrophils, endothelial cells) to secrete MMP-9 since high concentrations of glucose can be pro-inflammatory. This may also have contributed to the increased microhemorrhage with glucose.

After HS, the intestinal villi structure remains intact compared to its state in SAO models (Ikeda et al. 1998; Mitsuoka and Schmid-Schonbein 2000; Grossmann et al. 2002; Chang et al. 2012b). The macroscopic hemorrhagic lesions in which red cells enter the intestinal wall and the lumen are a common feature after shock (Chiu et al. 1970a; Manohar and Tyagi 1973; Haglund et al. 1975), but they are not formed if the intestine is flushed. Furthermore, the strong correlation between intestinal wall hemorrhage and proteolytic activity of the intestinal homogenate suggests that the digestive proteases play a role in the formation of these vascular lesions. Mucosal barrier damage is less severe if animals are fed an elemental diet restricting the pancreatic enzymes as opposed to normal nutrition (Bounous et al. 1967), which supports the idea that nutrients are essential for mucosal barrier protection (Chiu et al. 1970b; McArdle et al. 1972; Kozar et al. 2002), but proteases can be detrimental if this barrier breaks open during ischemia.

It has been previously shown that intestinal ischemia can result in transport of pancreatic proteases into the lamina propria (Chang et al. 2012a,b). The requirement for these enzymes to enter the intestinal wall (and subsequently for red blood cells to enter the intestinal lumen) is that the mucosal barrier must fail. Following HS, regardless of the state of flushing, epithelial cells were present in the lumen similar to previous reports, which indicate epithelial shedding into the lumen after intestinal ischemia (Bounous et al. 1967; Vakonyi et al. 1977; Robinson et al. 1981; Ikeda et al. 1998). This evidence suggests that even though the villi appear intact, openings in the barrier occur as epithelial cells are shed together with their attached mucin, providing pathways for pancreatic enzymes and luminal contents to enter the intestinal wall.

As pancreatic proteases may promote further mucosal barrier failure by cleaving interepithelial adhesion proteins, epithelial junctional protein levels were investigated. The presence of luminal content did not affect the levels of the tight junctional protein occludin. However, the adherence junctional protein E-cadherin was degraded predominantly in the nonflushed animals, which is consistent with previous evidence that E-cadherin degrades during intestinal ischemia, possibly by the digestive enzymes (Chang et al. 2012a,b).

Pancreatic proteases are not the only proteases entering or activated in the intestinal wall during HS. When whole intestines (wall plus lumen) of No-HS and HS-NF animal intestines were homogenized with their luminal contents the majority of the caseinolytic activity is derived from luminal pancreatic enzymes (Penn et al. 2007; Penn and Schmid-Schonbein 2008), which is expected to be similar between the two groups. However, the HS-NF animals exhibited higher levels of caseinolytic activity suggesting there may exist an additional source of proteases present in the HS-NF animals. As MPO activity also increased in intestinal homogenates in the HS-NF animals indicating neutrophil accumulation, neutrophil-derived elastase or MMPs could be an additional source of proteolytic activity. Alternatively, pancreatic proteases may convert pro-MMPs (or other proteases) into active MMPs (Duncan et al. 1998; Rosario et al. 2004) yielding a net increase in protease activity.

Studies using enteral treatment with the serine protease and lipase inhibitor ANGD in the small intestine have shown reduced microhemorrhages in the intestine and improved outcomes in experimental shock (Mitsuoka and Schmid-Schonbein 2000; Doucet et al. 2004; Shi et al. 2004; DeLano et al. 2013), which supports the hypothesis that digestive enzymes or their products are responsible for the lesions. If the pancreatic enzymes enter the wall but are inactive, they would be unable to trigger a proteolytic activation cascade of MMPs or degrade epithelial proteins. It should be noted that ANGD is solubilized in a glucose solution in those studies, and glucose itself serves to attenuate disintegration of the epithelial barrier during intestinal ischemia (Chiu et al. 1970a,b; Mirkovitch et al. 1975; Kozar et al. 2002). However, failure of the epithelial barrier alone does not appear to be sufficient to cause microhemorrhages in the intestine as there were no hemorrhages in HS-F animals despite the lack of a source of metabolic energy in the lumen of the intestine.

Development of microhemorrhages also requires that the endothelial cells lining the blood vessels in the intestine must fail. This may occur by a number of possible mechanisms. Pancreatic proteases, activated MMPs, or neutrophil elastase may digest the basement membrane and endothelial cell adhesion proteins allowing red cells to escape into the interstitial space (Allport et al. 2002; Hu et al. 2005). The proteases may cleave extracellular matrix or dietary proteins into bioactive peptides that increase vessel leakage, or cleave fatty acid binding proteins allowing unbound free fatty acids to act as detergents destroying endothelial cells (Penn and Schmid-Schonbein 2008; Davis 2010; Qin et al. 2012). Unbound free fatty acids may also lyse erythrocytes (Lujan and Bronia 1994), which could allow free hemoglobin to escape into the lumen (potentially bypassing the need for extracellular matrix or basement membrane breakdown). Bioactive peptides or free fatty acids may also be proinflammatory (Badwey et al. 1984; Ferrante et al. 1994; Penn et al. 2007, 2012) contributing to the neutrophil accumulation.

Although bacteria in the gut can be a potential injury mechanism for the intestine (Yale 1969; Musemeche et al. 1986; Deitch et al. 1991), they tend to be distributed throughout the gut with higher levels in the ileum regions compared to the jejunum (Salanitro et al. 1978; Fryer et al. 1996). However, the intestinal injury was greatest at locations where food boluses were present and where digestive enzymes are most concentrated.

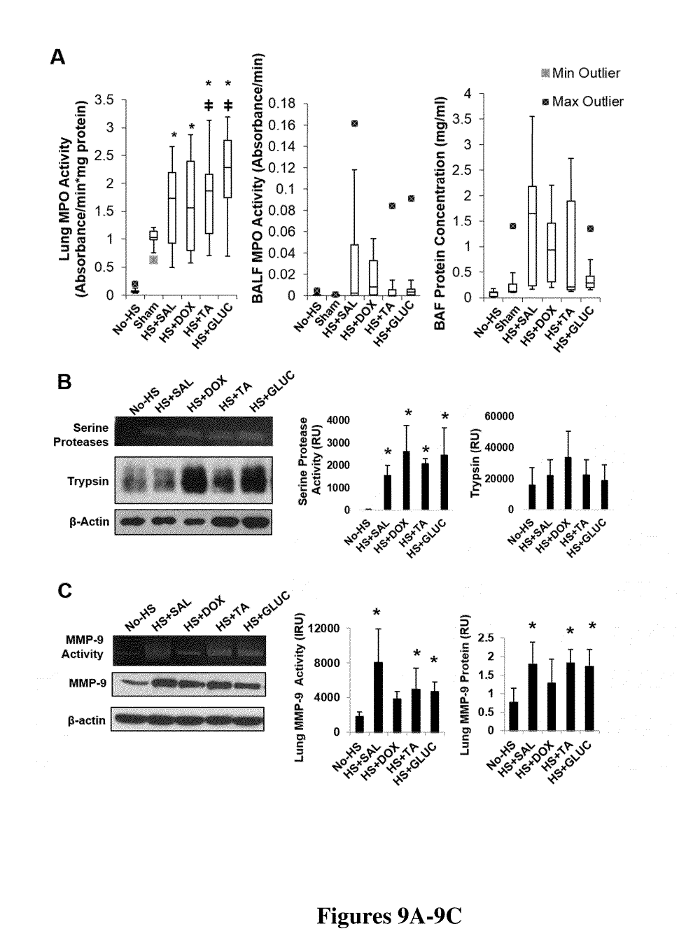

MMP inhibition and protein degradation—While pre-treatment with doxycycline reduced plasma MMP-9 activity by a specific peptic substrate and in the lung (FIG. 9C), and it moderately improved the intestinal gross morphology (FIG. 7B), cadherins in the lung still degraded and protein extravasation into BALF still occurred. Previous reports used doxycycline as an MMP inhibitor at comparable concentrations and documented improvement in vascular injury in organs such as the brain, lung, and kidney. The lack of protection of the lung with doxycycline suggests that in hemorrhagic shock, lung injury may be occurring by alternate and possibly multiple mechanisms. The ischemic conditions cause proteases beneath the surface of the endothelial cells to degrade the junctional endothelial proteins from the basal side of the vessel where doxycycline could insufficiently penetrate. The ischemia may also trigger internalization and degradation of the cadherins. Alternatively, MPO activity was significantly increased in the lungs after HS, and was not completely decreased by doxycycline (FIG. 9A), possibly due to the neutrophil and monocyte infiltration. MMP-9 or elastase released by these cells into microenvironments (and thus in part protected from circulating doxycycline) during and after extravasation could cleave junctional proteins and allow plasma protein leakage. Hemorrhage from lung microvessels likely does not require complete destruction of the entire vessel, but rather generation of openings to allow red cells and plasma to escape into the alveolar space.

Transport of pancreatic proteases into the periphery—The major translocation routes for luminal content from the intestine into the circulation can be directly via the portal venous circulation, the mesenteric lymph, or the peritoneal space and the lymphatics surrounding the peritoneum (Deitch 2010; Altshuler et al. 2012). Even before HS, trypsin and chymotrypsin were present in the plasma and remained unchanged after HS (FIGS. 8B and 9B). Serine protease activity bands, in contrast, were not present in the plasma before HS but increased afterwards. One possible explanation is that pancreatic proteases are constitutively released in zymogen form from the pancreas (we have evidence that non-active trypsin and chymotrypsin are also present in the mesenteric lymph vessels common to the pancreas and small intestine; Altshuler, et al., Physiological Reports, vol. 1, iss. 5, pp. 1-14, 2013), and may be activated in the lymph or plasma by even small amounts of active protease coming from the intestine during shock. This could result in a relatively small change in protein concentration in plasma with a much larger activity change. As zymogen trypsin and chymotrypsin secreted into the intestine are rapidly converted to their active forms, if the change after shock is due to activation, then it is likely that the zymogen enzymes present before shock originated directly from the pancreas rather than from the intestine, though the active enzymes responsible for their conversion during shock may originate from either organ (note that unlike MMPs, pancreatic serine zymogen proteases are not activated by gel zymography). Past proteomic studies of postshock mesenteric lymph have not reported trypsin or chymotrypsin in the lymph fluid, possibly because these enzymes are commonly used to digest samples prior to mass spectrum analysis (Fang et al. 2010; Zurawel et al. 2010). The significance of elevated protease activity in the lymph could have implications on lymphatic endothelial cell extracellular damage and degradation of proteins in the lymph fluid.

The pancreatic proteases were also present in all plasma samples before the onset of HS, but enzyme activity by zymography was undetectable. As electrophoresis would be expected to separate the enzymes from any plasma protease inhibitors, this supports the idea that these are zymogen proteases from the pancreas. However, the protease activity in gel zymography at low-molecular weight increased in post-HS plasma, regardless of the luminal contents, similar to previous findings (Altshuler et al. 2012). Possibly they are being activated during shock by pancreatic ischemia or in the lymph or plasma by small quantities of active enzymes from the intestine. Inhibition of these enzymes with ANGD and TLCK eliminated the bands, suggesting that they are pancreatic enzymes instead of MMPs or plasmin (Frederiks and Mook 2004).

Unlike plasma and lymph, which had no significant difference in serine protease activity between flushed and nonflushed groups following HS, lung serine protease activity was higher in the nonflushed case than the flushed case. It has been previously observed that not only trypsin activity, but also trypsin protein levels increase in the lung after HS (Altshuler et al. 2012). The lack of increase in the flushed group suggests that the enzymes are intestinal in origin. The lung is the first capillary bed encountered upon discharge of lymphatic fluid via the thoracic duct into the venous circulation. Therefore, active proteases from the lymph may become entrapped in the lung upon mixing with the venous blood rather than circulating systemically because plasma proteins can accumulate in the lung after shock (Staub 1981).

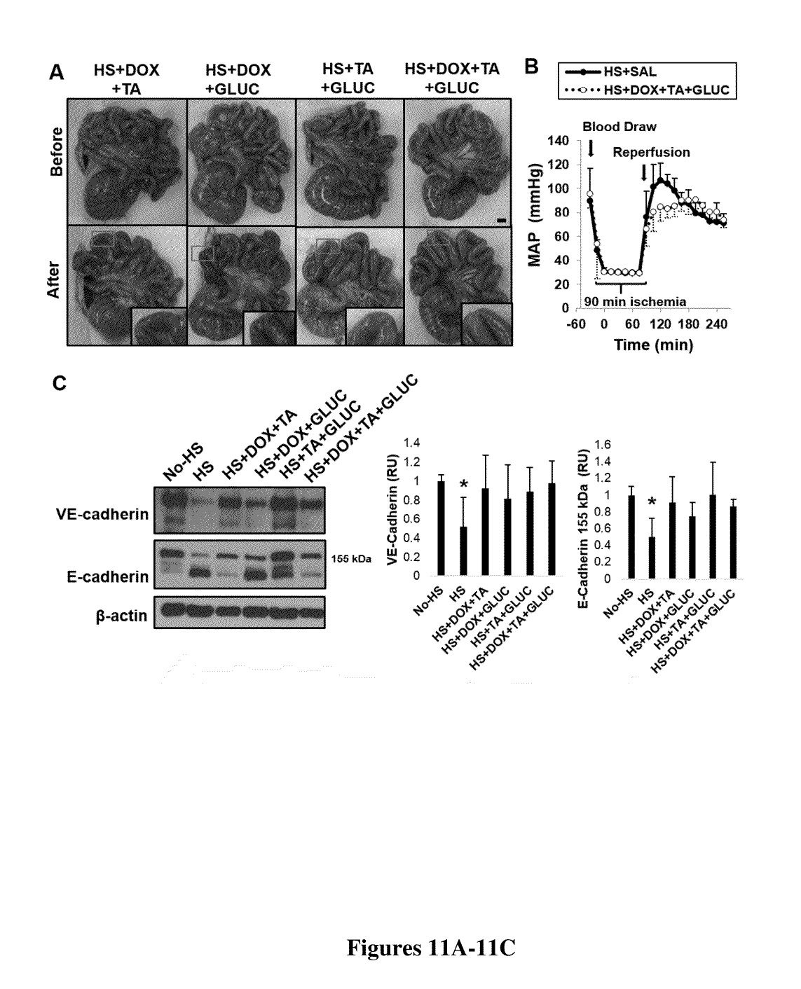

Plasma protease activity and membrane protein damage—Neither the decrease in plasma and lung MMP activity with doxycycline nor the increase in plasma serine or MMP activities with glucose correlated with lung E-cadherin or VE-cadherin degradation (glucose, in fact tended to preserve the cadherins), suggesting their degradation is largely independent of circulating protease levels. However, when gut morphology was protected by the use of combination treatments, the degradation of VE-cadherin and E-cadherin was attenuated (FIG. 11C), despite the fact that the protease levels remained elevated. This evidence suggests that cadherin integrity may be preserved by a more indirect mechanism than just lung protease inhibition (e.g., reducing transport of inflammatory mediators from the gut).

The intestine's MMP-9 levels increased only in the HS-NF animals and may be due to increased neutrophil accumulation in the wall, an observation that is supported by the increased MPO activity. Aside from the intestine, the MMP-9 protein levels in the plasma were elevated in groups with or without a flushed small intestine suggesting that MMP-9 secretion into the plasma did not depend on the luminal contents. MMP-9 levels and activity also increased in the lung during hypotension in both the HS-F and HS-NF animals. The active form of MMP-9 in the lung was nearly doubled in the HS-NF group compared to the HS-F group. The increase in MMP-9 activity may be due to activation by serine proteases transported into the lung (Duncan et al. 1998).

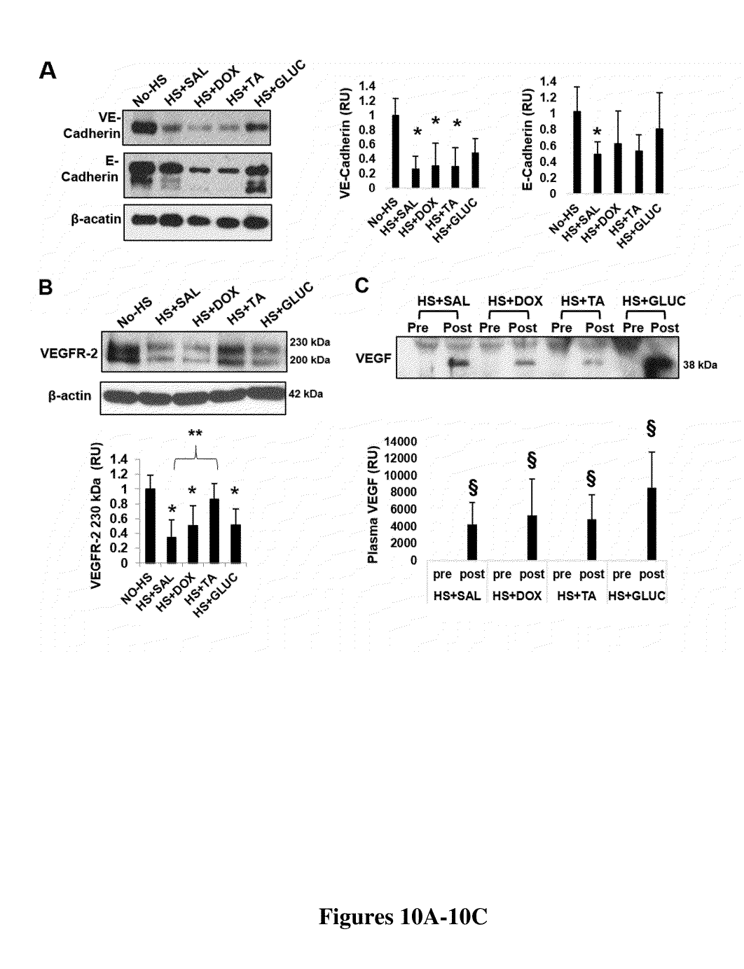

VEGFR-2 and the relationship between MMP-9 and circulating VEGF levels—After HS, VEGFR-2 receptor density decreased in the lung in HS+SAL animals but was preserved in the HS+TA group (FIG. 10B). Previously, 30 minutes of superior mesenteric artery occlusion of mice was not sufficient to reduce VEGFR-2 levels in the lung which suggests that the global ischemia is necessary to decrease lung VEGFR-2 levels. Tranexamic acid in the gut may be penetrating into the periphery during reperfusion and protect the surface density of the VEGFR-2 receptor by inhibiting proteases in the plasma. VEGFR-2 levels decreased even in lungs with low lung injury score in HS+GLUC or HS+SAL animals suggesting that lung injury and VEGFR-2 levels are not necessarily correlated.

There was a significant appearance of soluble VEGF in the circulation (FIG. 10C). Since plasma TIMP-1 levels remained unchanged (FIG. 8D), the unchecked upregulation of MMP-9 activity may be responsible for the degradation of the extracellular matrix that releases VEGF from the tissue into the circulation. MMP-9 can also post-process VEGF to different isoforms. It may be possible that the release of VEGF after ischemia is an early response to stimulate the endothelial cells to repair damaged vessels. The lack of VEGFR-2 receptors and the increase in VEGF ligand may cause an imbalance in the apoptotic/anti-apoptotic signaling cascade and warrants future investigations. The preservation of VEGFR-2 may in part explain why enteral tranexamic acid treatment significantly increases survival after HS.

These studies support the hypothesis that a major part of the organ injury in hemorrhagic shock is a proteolytically driven process. Doxycycline administration reduced the MMP activity in the plasma and provided intestinal protection, but was relatively ineffective at preventing protein degradation in the lung. Enteral tranexamic acid treatment reduced intestinal injury. In addition, enteral glucose provided metabolic support to the intestinal epithelium and preserved the mucosal barrier. Each of these treatments has select advantages. The combination of these treatments served to minimize intestinal injury and protein degradation in the lung following hemorrhagic shock and supports the notion of multiple mechanisms involving, at one stage or another, degrading proteolytic processes, which contribute to the progression of shock and multiple organ failure.

The increase in transmural permeability to small molecules during total ischemia of the small intestine occurs by a combination of epithelial shedding and MMP-derived proteolysis of the intestinal wall, both of which occur in the absence of luminal contents (e.g., food, digestive enzymes, bacteria, viruses, etc.). The rise in transmural permeability during ischemia was reduced by placement of glucose into the lumen of the intestine, as well as by administration of tranexamic acid or MMP inhibitors. Dual treatment preserved intestinal histology (both the overall villi structure and the connection between epithelium and lamina propria) after complete ischemia. Dual treatment, but not individual treatments, with glucose and tranexamic acid in a model of hemorrhagic shock prevented visible intestinal hemorrhage and stabilized blood pressure during reperfusion. The results provided herein suggest that prophylactic treatment with enteral glucose and/or MMP inhibition in trauma patients at high risk for intestinal ischemia may reduce intestinal permeability and transmural passage of luminal content to improve the outcome after ischemic injury.

Lung Inflammation—Although there was more MMP-9 and serine protease activity in the lungs in the HS-NF animals, the BALF protein levels and MPO activity did not differ between groups after HS. Enteral protease inhibition with ANGD has been shown to attenuate MPO infiltration into the lungs in a similar model of HS (Altshuler et al. 2012), which, combined with the findings presented herein, suggests that the protease inhibition is acting at sites distant from the intestinal lumen. Paradoxically, these same pancreatic protease inhibitors are not effective at attenuating organ injury if administered systemically instead of enterally (Deitch et al. 2003; Shi et al. 2004). This suggests that neutrophil activators may be arising from multiple locations in the body during HS, and that if either the intestine or the alternate source is ignored, neutrophils may become activated resulting in lung injury. Given that the pancreas contains proteases and during ischemia could generate many of the same mediators generated by the intestine, the pancreas may be an alternative source of inflammatory mediators (Kistler et al. 2000). This hypothesis is supported by reduced lung damage in studies that ligate the mesenteric lymph vessel as a carrier of mediators from both intestine and pancreas (Deitch 2010). Intravenous ANGD is an approved treatment for pancreatitis in Japan, supporting the idea that enteral ANGD, if absorbed into the circulation or leaked into the peritoneal space, could reduce damage and mediator release in the pancreas.

VE-cadherin, an adhesion protein expressed on lung endothelial cells (Shasby 2007), occludin, a tight junction protein present on both endothelial and epithelial cells in the lung, and both isoforms of the endothelial growth receptor VEGFR-2 were reduced after HS regardless of flushing the luminal contents. E-cadherin, another endothelial adhesion protein also (nonsignificantly) decreased after shock, and there was a significant increase in a smaller molecular weight form of the protein suggesting cleavage by a protease. Neutrophils accumulate in the lung during shock and could therefore contribute to the loss and/or cleavage of these proteins, as neutrophil elastase and MMPs have been documented to contribute to the destruction of cadherins (Carden et al. 1998; Navaratna et al. 2007), possibly as part of neutrophil transmigration. The loss of any or all of the proteins tested here could contribute to increased lung permeability. The reduction of the endothelial survival receptor VEGFR-2 could impair vascular repair following the ischemic injury (Holmes et al. 2007). However, as all of these proteins were degraded regardless of the presence of luminal content, the mechanism is likely independent of the proteases present in the lumen of the small intestine and more dependent on MMPs activated in the lung during ischemia. Though damage was independent of protease activity at the end point for this study (3 h postreperfusion), it is possible that the accumulated protease activities in the lung could cause increased damage at later time points or to a greater extent in other animal models or humans. Additionally, other proteins in the lung may also be degraded and could be affected by the increase in proteolytic activity from the luminal contents present in the intestine.

The following examples are intended to illustrate but not limit the invention.

### EXAMPLE 1

Male Wistar rats (body weight between 250-400 g, Harlan, Indianapolis, Ind.) for the intestinal ischemia model and male Sprague Dawley rats (body weight between 255-435 g, Harlan) for the hemorrhagic shock model were allowed food and water ad libitum prior to surgery. All rats were administered general anesthesia (xylazine, 4 mg/kg; ketamine 75 mg/kg IM.) and euthanized with B-Euthanasia (120 mg/kg).

Intestinal Ischemia—Since intestinal properties are non-homogenous, the transmural permeability was investigated over the entire length of the jejunum and ileum. Due to physical constraints of the small intestine anatomy, it is not feasible to simultaneously analyze permeability from multiple segments in vivo. Therefore, an ex vivo approach similar to previously published studies was designed to measure permeability along the length of the small intestine.

A midline incision was made to expose the intestine in anesthetized rats. The proximal end of the jejunum (approximately 5 cm distal from the ligament of Treitz) was cannulated with a female luer to barb tube connector (⅛″ inner diameter). The intestine was removed and placed in saline immediately before euthanasia. In order to focus the analysis on permeability of the intestinal wall during ischemia in the absence of luminal contents (including the digestive proteases or cytotoxic factors from food), the lumen of the intestine was flushed with 40 ml saline using pulsatile pressure. Visual examination confirmed all solid contents were removed. Next, the intestine was cut into eight equal length segments (˜8 cm) sequentially ordered from the proximal jejunum (position 1) to the distal ileum (position 8). Both ends of each segment were cannulated with Female Luer to Barb tube connectors (thus allowing entry and exit points from the intestinal lumen to be sealed completely), secured with 4-0 suture, and the exterior portion of one adaptor on each segment sealed with clay.

To determine the transmural permeability of the intestine, all 8 segments from each animal were filled with a low molecular weight tracer as a sensitive measure of early barrier breakdown prior to the initiation of ischemia, fluorescein (332 Da M.W., 20 μg/ml from 5 mg/ml stock in ethanol; 0.308 osm/L; Sigma-Aldrich, St. Louis, Mo.) mixed with saline, glucose (100 mg/ml in saline; 0.550 moles/L; 0.863 osm/L, Sigma-Aldrich), the non-metabolizing glucose analog mannitol (100 mg/ml in saline; 0.550 moles/L; 0.863 osm/L), the serine protease inhibitor tranexamic acid (31 mg/ml in saline; 0.200 moles/L; 0.508 osm/L, Sigma-Aldrich), the MMP inhibitor doxycycline hyclate (1 mg/ml in saline; 1.955×10⁻³ moles/L; 0.310 osm/L, Sigma-Aldrich), or the MMP inhibitor GM 6001 [38] (1 μg/ml in saline from 1 mg/ml stock in DMSO; 2.57×10⁻⁶ moles/L; 0.308 osm/L, Millipore, Billerica, Md.). Concentrations were chosen to match those previously used in in vivo shock studies (e.g., the tranexamic acid concentration is in the range that was previously used to inhibit enteral trypsin).

300 μl of sample were added to each intestinal segment (this volume does not fully inflate or stretch the intestinal tissue) through the tubing adaptor before sealing with clay. Sealed segments were rinsed in saline, individually placed in 15 ml conical tubes containing 6 ml saline, and incubated at 37° C. for 2 hours to simulate ischemic conditions. The exterior solution for each position was sampled at 0, 30, 60, 90, and 120 minutes and loaded in duplicate (75 μl/well) into a 96 well plate (black-sided flat bottom polystyrene, Corning, New York, N.Y.). Plates were read in a plate reader (FilterMax F-5 Multi-mode, Molecular Devices, Sunnyvale, Calif.) for concentration of fluorescein (excitation 494/emission 521) to measure diffusion across the wall of the intestine.

After the two-hour ischemic incubation, intestinal pieces from segments 2 (jejunum) and 7 (ileum) were embedded in O.C.T. (Tissue Tek, Torrance, Calif.) and snap frozen in t-methyl butane in liquid nitrogen. Intestine samples from segments 2 and 7 (for protease analysis) and segment 7 (for immunoblots) were snap frozen and stored for subsequent homogenization.

The small intestines of a separate group of animals were used as pre-ischemic controls for morphology and the corresponding regions of tissue were embedded in O.C.T. and frozen. As a non-ischemic control for permeability, a segment of distal ileum (approximating segment 7 which had the greatest permeability in the ex-vivo ischemic case) from a separate group of animals was cannulated, flushed, filled with saline with fluorescent markers, as described above, and immersed in a small saline bath, but the blood supply to the segment was left intact. The animals were maintained under general anesthesia for 2 hours, and the samples of exterior fluid from the saline bath were measured and adjusted for volume dilution in the bath.

Permeability Analysis—To determine the fluorescein transport across the ischemic intestinal wall, the fluorescence at time 0 was subtracted from fluorescence values at later time points to correct for background, and fluorescence values were corrected to account for the volume reduction due to sampling during the experiment. The RFU measurements were converted to equivalent moles by using the linear relationship between concentration and fluorescein fluorescence (not shown, R²=0.999). The transport of fluorescein across the wall (measured in moles, per time) was computed as the change in fluorescence over a 30 min interval divided by 30 min. Individual positions were excluded if the fluorescence measured at time 0 exceeded a pre-selected minimum threshold, suggesting a torn specimen or a specimen with incompletely sealed ends (this occurred 6 times out of a total of 192 intestinal segments).

Hemorrhagic Shock Model—Hemorrhagic shock (HS) was used to study the ability of treatments, shown in the ex vivo portion of the study to preserve the barrier and protect the intestine in the absence of luminal content, to prevent intestinal injury after ischemia/reperfusion injury in vivo when luminal content is present.

Following general anesthesia, the femoral artery and vein were cannulated. Systolic, diastolic, heart rate, and mean arterial pressure (MAP) were recorded throughout the procedure using LabChart (AD Instruments, Dunedin, New Zealand). Rats were randomly divided into the following groups: No-HS (N=6), HS+Saline (N=6), HS+Glucose (N=7), HS+Tranexamic Acid (N=7), and HS+Tranexamic Acid+Glucose (N=4). No-HS animals were cannulated and immediately sacrificed for tissue collection.

In the HS groups, anesthetized rats were subjected to laparotomy and the intestine was exposed, and a “Pre-HS” image was captured. Using a 5 ml syringe (Becton Dickinson), intraluminal treatments of either saline, 10% glucose in saline, 200 mM tranexamic acid in saline, or the combination of 10% glucose with 200 mM tranexamic acid in saline was warmed to 37° C. in a water bath before injecting each treatment in two to three sites along the length of the intestine from the jejunum to the ileum (10.7±1.0 ml; mean±SD). Extra caution was applied to avoid over-inflation of the intestine. After injection, the intestine was carefully returned to the peritoneal cavity and the wound was covered with moist gauze and plastic wrap to keep the animal warm.

To prevent clotting in catheters and shed blood, animals were heparinized (1 U/ml i.v., assuming 6% blood volume per body weight) prior to inducing hemorrhagic shock. Blood was removed through the femoral venous catheter (0.4 ml/min) until the MAP reached 30 mmHg. MAP was maintained at 30 mmHg by withdrawal/return of blood over the 90-minute ischemia period. At the end of ischemia, the shed blood was returned to the animal (0.5 ml/min). The animal was kept anesthetized and was observed for 3 hours. Gross morphology images of the intestine were captured, “Post-HS”

At the end of reperfusion, segments of intestine (jejunum; 10 cm from ligament of Treitz) were snap frozen for homogenization and embedded in O.C.T. (Tissue Tek).

Morphological Analysis and TUNEL Labeling—Intestinal segments frozen in O.C.T. were cut into 5 μm sections. Sections were fixed using ice-cold methanol (8 min at −20° C.) and immediately washed four times in distilled water. Nuclei were stained by incubating sections in freshly mixed Weigert's Iron Hematoxylin A and B (Electron Microscopy Science, Hatfield, Pa.) (10 min) followed by rinsing thoroughly with water. Collagen fibers were labeled by incubating (2 min) in Van Gieson's Solution (Electron Microscopy Science).

To assess the level of apoptosis after ischemia, in situ terminal transferase dUTP nick end labeling (TUNEL) labeling was completed using a kit (Trevigen, Gaithersburg, Md.) according to manufacturer's instructions. Sections were counterstained with 0.05% toluidine blue in 1% boric acid to contrast 3,3′-diaminobenzidine (DAB) positive nuclei (brown) from negatively stained nuclei (blue).

Prior to mounting, all slides were dehydrated (70, 95, 100% ethanol) and cleaned with xylene. After air-drying overnight, sections were mounted using Hard Set Mounting Media (Vector Laboratories, Burlingame, Calif.). Digital images were captured with a 20× objective (numerical aperture 0.5) and digitally montaged together after background subtraction.

The separation of the mucosal epithelial layer was quantified for groups that had intact villi following the two hour ischemic period. Images were blinded and the distance between the mucosal epithelial layer and the lamina propria was measured over five lengths across a minimum of 6 villi.

Gelatin Gel Zymography—Segments 2 and 7 from pre-ischemic intestines were homogenized (0.1 g tissue/ml homogenization buffer; PBS pH 7.4 with 0.5% hexadecyltrimethyl bromide) and centrifuged at 1.4×10⁴ g for 20 minutes. Supernatants were stored at −80° C. until samples were processed.

To assess the proteolytic activities of individual proteases in intestinal tissue samples, sample volumes of 1 μl (with 2 μg of protein) were separated by gel electrophoresis in SDS-PAGE gels containing 80 μg/ml gelatin. Gels were renatured by four 15 min washes with 2.5% Triton X-100 and incubated overnight at 37° C. in developing buffer (0.05 M Tris base, 0.2 M NaCl, 4 μM ZnCl₂, 5 mM CaCl₂.2H₂O). After incubation, gels were fixed and stained (50% methanol, 10% acetic acid, 40% water, and 0.25% Coomassie blue solution) for three hours before de-staining in water. The molecular weights of the proteases were estimated by use of a standard protein ladder (Invitrogen). Gels were digitized and bands were analyzed by densitometry in ImageJ (rsbweb.nih.gov/ij/).

To determine which of the enzymes present in the intestinal wall are directly inhibited by tranexamic acid, some selected gels were renatured and developed with 20 mM tranexamic acid in their renaturing and developing buffers. These gels were compared to a concurrently run gel processed normally. Additionally, to confirm MMPs were being inhibited by tranexamic acid, doxycycline (1 mg/ml) or GM-6001 (1 ug/ml) were added to the renaturing and development buffer for select gels.

GM-6001 (N-[(2R)-2-(hydroxamidocarbonylmethyl)-4-methylpentanoyl]-L-tryptophan methylamide) is also known as galardin or ilomastat, and is a broad-spectrum matrix metalloproteinase inhibitor. GM6001 is a member of the hydroxamic acid class of reversible metallopeptidase inhibitors. The anionic state of the hydroxamic acid group forms a bidentate complex with the active site zinc.

MPO Activity—Myeloperoxidase (MPO) activity in the intestine as a measure for neutrophil accumulation was selected as an index of reperfusion injury after hemorrhagic shock. Segments from the jejunum (about at the position of segment 2 in the ex vitro study) were homogenized with their native luminal contents (PBS pH 6.0 in 0.5% HTAB). 40 μl of 2 mg/ml intestine homogenate was added to 180 μl of PBS (pH 6.0) mixed with 0.167 mg/ml o-dianisidine dihydrochloride (Sigma-Aldrich) in duplicate and 0.001% H₂O₂ (w/v).

Absorbance was measured kinetically at 450 nm every 5 minutes for 1 hour at 37° C. As negative controls, 180 μl of PBS (pH 6.0) was added to 20 μl of sample and the absorbance values of these samples were subtracted from the measurements with the substrate. The change in absorbance was linear within this period, and MPO activity is presented as the change in absorbance per minute per milligram of protein.

Immunoblotting—To determine whether selected epithelial barrier proteins were degraded during ischemia, pre- and post-ischemic intestinal tissue homogenates (segment 7) were analyzed for relative levels of mucin 13, occludin, and E-cadherin.

Tissues were homogenized for immunoblot analysis in buffer (CelLytic, Sigma-Aldrich) with protease inhibitor cocktail (HALT, Thermo Scientific, 1:100 dilution) and centrifuged (1×10⁴ g, 20 min). Protein concentration in supernatants was determined (BCA, ThermoScientific). To separate proteins by gel electrophoresis, 30 μg of protein/well were loaded in 8 or 12% resolving gels with 4% stacking gels and transferred to nitrocellulose membranes (Bio-Rad, Hercules, Calif.). Membranes were blocked for one hour with either 5% bovine serum albumin (BSA) or 5% nonfat milk in tris-buffered saline with 0.5% Tween-20 (TBS-T). Primary antibodies diluted in 1% BSA or 1% nonfat milk against mucin 13 (1:1000, extracellular domain, sc-66973 Santa Cruz Biotechnology, Santa Cruz, Calif.), E-cadherin (1:1000, intracellular domain, 33-4000, Invitrogen), occludin (1:1000, intracellular domain, 33-1500, Invitrogen), pancreatic trypsin (1:1000, sc-137077 Santa Cruz Biotechnology), or β-actin (1:1000, sc-130301, Santa Cruz Biotechnology) were incubated overnight at 4° C. After incubation, membranes were washed with TBS-T (3×, 10 min) before addition of goat anti-rabbit or rabbit anti-mouse secondary antibodies (Santa Cruz Biotechnology) diluted 1:1000 in TBS-T. Following secondary antibody incubation (60 min), the membranes were washed with TBS-T (3×) and developed.

Statistical Analysis—Results in the severe ischemia ex vivo model are shown as mean±standard error of mean (SEM) (N=3-6/group) to display with greater clarity the time courses for the tracer transport. Two factor ANOVA was used to compare between positions (1-8) over time within each treatment group followed by Tukey post hoc analysis to compare each position over time and each position to position 2 (which typically showed the lowest permeability). To compare the saline group to the treatment groups, the data were fit with polynomial equations (third order polynomials gave the best data representation). Inhibitors' equations were compared to saline's equation by the F-test. Single factor ANOVA analysis followed by Tukey post hoc test was used for immunoblot comparisons. Paired t-test was used to compare protease activity in jejunum and ileum segments. Statistical analysis was performed using Origin software (Northampton, Mass.). Results for hemorrhagic shock are presented as mean±standard deviation (SD).

Results—Transmural permeability is attenuated with metabolic support from glucose—During ischemia in the absence of treatment, fluorescein transport across the intestinal wall was positive with increasing fluorescence at all locations along the intestine for every time interval (FIG. 1A). The average fluorescein transport from ischemic intestines containing normal saline increased with position along the length of the intestine from the jejunum to the ileum (though position 1 at the proximal jejunum generally had a higher transport than position 2) (FIG. 1A). Glucose administration into the lumen of the intestine drastically reduced the fluorescein transport across the wall of the intestine even after 2 hours (FIG. 1A). Ischemic conditions in the saline group led to destruction of the intestinal villi structure both in the jejunum and the ileum that was attenuated in the glucose group (FIG. 1B). This was attenuated in the glucose group, however, separation between the lamina propria and the mucosal epithelial layer was still observed, as was internal damage to the villi.

Pre-ischemic control intestines had little apoptosis except near the tips of the villi (FIG. 1C) while saline-treated ischemic intestines showed extensive apoptosis in cells in the villi as well as cells detached from the villi (nuclei are visible despite loss of villi structure). Glucose treated intestines had little apoptosis in the villi, though TUNEL labeling similar to that in the saline group was present in the muscle layer (FIG. 1C). After severe intestinal ischemia, mucin 13 bound to the epithelium and occludin decreased in the saline group but were preserved in the presence of luminal glucose (FIG. 1D). E-cadherin decreased irrespective of the presence of glucose (FIG. 1D), suggesting it is not involved in the preservation of the epithelial barrier to fluorescein during severe ischemia.

To confirm the beneficial effects of glucose were not a consequence of increased osmolality, mannitol, a non-metabolizable sugar, was substituted for glucose. Mannitol neither prevented fluorescein penetration nor preserved villi structure (not shown), in line with previous findings.

When the fluorescein transport rate was measured in-vivo in an isolated non-ischemic ileum (segment 7) with intact perfusion, a small reverse convective flux of fluid from the saline bath into the vasculature and lymphatics of the intestine was detected (resulting in fluorescein influx of 0.05±0.06 nM/min at the 90-120 min time point, N=3 rats/group). In contrast, in the ischemic intestine (also in segment 7) only efflux of fluorescein was seen in the ex vivo saline group (4.83±0.36 nM/min, N=6 rats/group). The villi in the vascularized control were intact at the two-hour collection time point (not shown).

Protease activities of intestinal homogenates—The proteolytic activity profile between the jejunum (segment 2) and ileum (segment 7) differed significantly (FIG. 2A) as determined by gelatin gel zymography in pre-ischemic control tissue homogenates (FIG. 2A). Many of these bands were reduced by addition of tranexamic acid to the renaturing and developing solutions (FIG. 2A). Though reported primarily as a plasmin inhibitor, tranexamic acid was nevertheless able to significantly reduce the bands that formed in the ileum around 220 kDa corresponding to MMP-9 dimers, pro-MMP-2, MMP-2, and the 50 kDa band corresponding to either MMP-1 or MMP-3, but had no significant effect on the 20 kDa serine proteases (FIG. 2B). Protease activity bands still formed with equal intensity in the ileum even if only the ileum were flushed indicating that the increased activity in the ileum is not arising from the passage of jejunal contents (not shown). As positive controls, gels were renatured with either doxycycline or GM-6001 and all bands except for the serine protease bands (˜20 kDa) were reduced or eliminated and serine protease bands remained in all gels for all samples (data not shown).

Intestine tissue homogenates from the 2 hr ischemic saline treated group had no change in the protease activity profile by gelatin gel zymography compared to the pre-ischemic intestinal homogenates (results not shown).

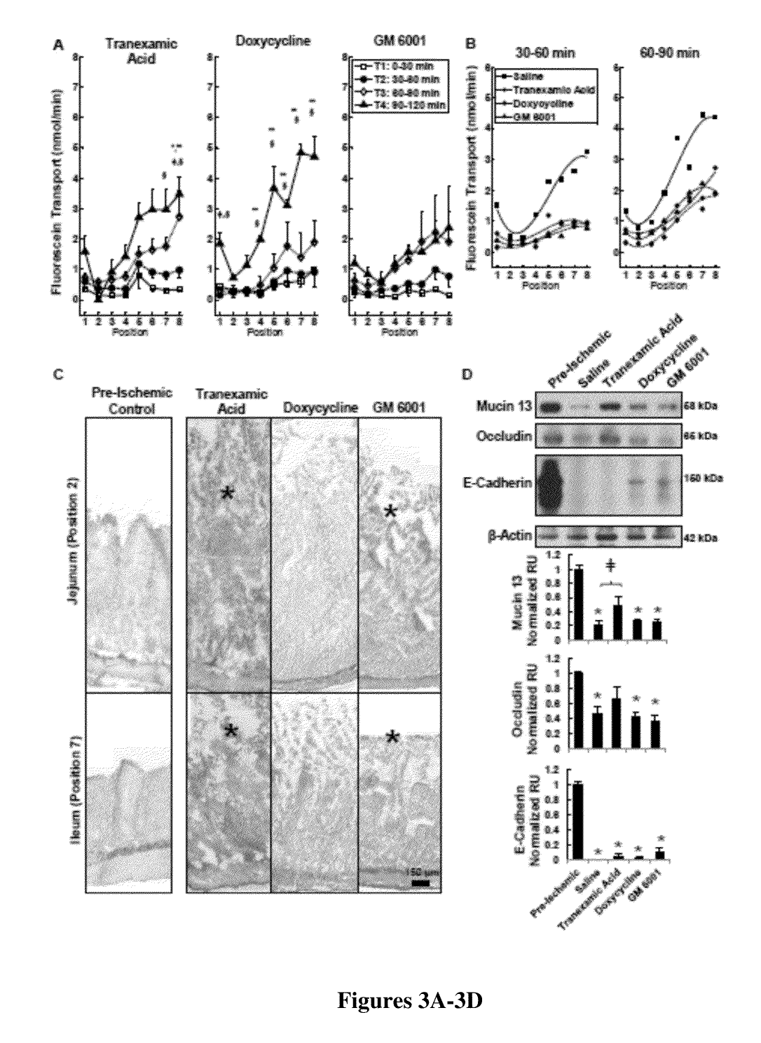

MMP inhibition reduces transmural permeability—Inhibition with tranexamic acid or the broad-spectrum MMP inhibitors doxycycline and GM 6001 prevented significant increases in fluorescein transport at early time points (FIG. 3A). The transport rates in the doxycycline and GM 6001 group closely matched the tranexamic acid group in the 30-60 min and 60-90 min periods and were significantly decreased compared to the saline treated intestines (FIG. 3B). However, in the 90-120 min period, GM 6001 was more effective at reducing the transmural permeability compared to either doxycycline or tranexamic acid (not shown). The villi in the jejunum and ileum were not intact after 2 hrs of ischemia (FIG. 3C).

In the ex vivo model, neither doxycycline nor GM 6001 were effective at preserving mucin 13, occludin, or E-cadherin breakdown at the two hour time point, but tranexamic acid was able to preserve mucin 13 levels and prevented a significant decrease in occludin. This suggests that MMPs are not directly involved in the earliest breakdown of the mucosal barrier during severe ischemia (i.e., their early benefits to transmural permeability could reflect preservation of other barriers to diffusion such as ECM proteins or cell/matrix or cell/cell adhesions in the muscle layer). Since mucin plays a role in protection of the intestine when luminal content is present, tranexamic acid in this model may be a better choice for intestinal protection in vivo than an inhibitor that targets MMPs alone.

Hemorrhagic Shock with enteral glucose or tranexamic acid—Following this ex vivo analysis, the interventions most effective at minimizing intestinal destruction were applied in the clinically more relevant model of hemorrhagic shock with an unflushed intestine. Tranexamic acid was chosen over GM 6001 because it was able to preserve mucin 13, reduce the separation of the mucosal epithelial layer from the lamina propria, and does not require solubilization in an organic solvent (DMSO).

For the first part of this study, the lumen of the intestine was pre-treated before the onset of hemorrhagic shock with single treatments only. After hemorrhagic shock, all animals regardless of pretreatment with saline, glucose, or tranexamic acid had lesions form in the jejunum (FIG. 4A). The neutrophil accumulation in the jejunum was elevated after HS in all groups (FIG. 4B). The MAP decreased linearly during 2 hours of reperfusion in all cases after HS at rates of −0.32±0.12 mmHg/min, −0.21±0.13 mmHg/min, −0.27±0.16 mmHg/min for HS+Saline, HS+Glucose, and HS+Tranexamic Acid, respectively. The glucose group had on average a lower starting pressure and final pressure due to the parasympathetic activity of glucose.

Histological sections of the intestine after hemorrhagic shock showed no destruction of the villi, unlike the severe ischemic model. There was no mucin 13 or occludin degradation after shock in intestinal homogenates confirming the inherent differences between the ex vivo model and the hemorrhagic shock models (not shown). The effect of combining the metabolic support with protease inhibition treatment was then examined in both the ex vivo severe ischemia and hemorrhagic shock models.

Gut protection with tranexamic acid+glucose after hemorrhagic shock—The combination of tranexamic acid+glucose and GM 6001+glucose (FIG. 5A) served to maintain low permeability throughout the two-hour ischemic period and did not behave differently than glucose alone. Because of the glucose, the intestine villi also remained intact following 2 hours of intestinal ischemia (FIG. 5B). However, unlike glucose alone, the epithelial layer stayed adhered to the lamina propria in the jejunum significantly better when tranexamic was combined with glucose. GM 6001+glucose resembled the glucose case but did not improve the protection against the separation from the lamina propria. The separation of the mucosal epithelial layer in the jejunum was significantly reduced compared to the glucose group when tranexamic was added (FIG. 5C).

Since the dual treatments appeared to maintain an epithelial layer and prevent internal tissue degradation, we tested the combination of glucose and tranexamic acid in hemorrhagic shock. Tranexamic acid was chosen over GM 6001 because it was able to preserve mucin 13, reduce the separation of the mucosal epithelial layer from the lamina propria, and does not require solubilization in an organic solvent (DMSO). The intestine appeared healthy in all animals prior to the onset of shock with no evidence for hemorrhage or distensions (FIG. 6A). The majority of the lesions in the jejunum region were reduced if tranexamic acid+glucose were administered simultaneously rather than independently (FIG. 4A and FIG. 6A).

It was sought to measure the degree of neutrophil infiltration into the intestine after shock and found that the animals treated with tranexamic acid and glucose did not have significantly lower MPO activities, though there was one outlier with high MPO activity (FIG. 6B). The animal with the greatest MPO activity in the HS+Tranexamic Acid+Glucose (outlier) also had macroscopic hemorrhages similar to the HS+Saline animals.

The MAP in the reperfusion phase shows that the HS+Saline animals had a higher initial pressure at the start of reperfusion that decreased at a faster rate compared to the HS+Tranexamic Acid+Glucose treated animals (FIG. 6C). Comparing the average regression between the groups of the MAP drop from the last 2 hours of reperfusion, HS+Saline animals decreased at a rate of −0.32±0.12 mmHg/min (mean±SD), which was significantly faster (p<0.005 by t-test) than the HS+Tranexamic Acid+Glucose animals which decreased at a slower rate of −0.07±0.08 mmHg/min (mean±SD).

### EXAMPLE 2

Adult male Sprague Dawley rats (mean±standard deviation (SD) body weight 340±60 g, N=64, Harlan, Indianapolis, Ind.) were allowed food and water ad libitum prior to surgery. Rats were administered general anesthesia (xylazine, 4 mg/kg; ketamine 75 mg/kg IM) and remained anesthetized throughout the experiment. At the termination of experiments, rats were euthanized by infusion of B-Euthanasia IV (120 mg/kg). The femoral artery and vein were cannulated. Systolic, diastolic, heart rate, and mean arterial pressure (MAP) were recorded throughout the procedure using LabChart (AD Instruments, Dunedin, New Zealand).

Hemorrhagic Shock (HS) with Removal of Luminal Contents—Animals were grouped into no-HS (No-HS), HS with intestinal luminal contents flushed (HS-F), and HS without intestinal flush (HS-NF). No-HS animals were immediately sacrificed for tissue collection following cannulation. All other animals were subject to laparotomy before the intestine was exposed and gross morphology was photographically recorded. Enteral treatments were administered to the intestine before returning to the peritoneal cavity (Table 1). IV/IP injections were delivered following the intestinal injections.

TABLE 1
Treatments administered before hemorrhagic shock.

| | Intraluminal* | IV^{†} | IP^{‡} |
|---|---|---|---|
| No-HS | N/A | N/A | N/A |
| Sham | Saline | Saline | Saline |
| HS | N/A | N/A | N/A |
| HS + SAL | Saline | Saline | Saline |
| HS + DOX | Saline | Doxycycline | Doxycycline |
| | | (5 mg/kg body | (10 mg/kg body |
| | | weight) | weight) |
| HS + TA | Tranexamic Acid | Saline | Saline |
| | (200 mM in | | |
| | saline) | | |
| HS + GLUC | Glucose | Saline | Saline |
| | (100 mg/ml in | | |
| | saline) | | |
| HS + DOX + | Tranexamic Acid | Doxycycline | Doxycycline |
| TA | (200 mM in | (5 mg/kg body | (10 mg/kg body |
| | saline) | weight) | weight) |
| HS + DOX + | Glucose | Doxycycline | Doxycycline |
| GLUC | (100 mg/ml in | (5 mg/kg body | (10 mg/kg body |
| | saline) | weight) | weight) |
| HS + TA + | Tranexamic Acid | Saline | Saline |
| GLUC | (200 mM) and | | |
| | Glucose | | |
| | (100 mg/ml in | | |
| | saline) | | |
| HS + DOX + | Tranexamic Acid | Doxycycline | Doxycycline |
| TA + GLUC | (200 mM) and | (5 mg/kg body | (10 mg/kg body |
| | Glucose | weight) | weight) |
| | (100 mg/ml in | | |
| | saline) | | |

*Intraluminal treatments were injected in 2-3 places after removal from the peritoneal cavity. Average injection was 10.8 ± 1.6 ml; mean ± SD.
^{†}IV injections were completed after intestine was replaced to peritoneal cavity. Doxycycline injection of 10 mg/ml.
^{‡}IP injections were completed after intestine was replaced to the peritoneal cavity. Doxycycline injection of 10 mg/ml.

The proximal jejunum (˜5 cm distal from the ligament of Treitz) was carefully cut between intestinal blood vessels and each end was cannulated with Female Luer to Barb tube connectors and secured with 4-0 suture. The connector cannulated to the duodenum was sealed with clay to prevent entry of new pancreatic proteases into the jejunum and ileum or peritoneal space after the intestine was flushed. Next, the distal ileum located three centimeters from the cecum was cut in order to insert another clay sealed connector into the intestine on the distal side (closest to the cecum) and secured with suture. The proximal end was not cannulated and sealed until after the luminal contents were flushed.

To remove the luminal contents, a syringe filled with 40 mL of saline at 37° C. was connected to the adaptor attached to the proximal jejunum, and the luminal contents were flushed distally with saline using pulsatile pressure. The contents exiting the intestine through the distal ileum were collected and discarded. Using this technique, all visible luminal content was removed from the intestine, and the last ˜15 mL of saline exiting the intestine were clear of color and absent of solid food residues. Following the intestinal flush, the final connector in the distal ileum was inserted and secured with suture. All connectors were sealed with clay. The exterior of the intestine was rinsed with warm saline (37° C.) and placed into the peritoneal cavity. HS-NF animals received the same manipulation and placement of connectors in the intestine, but the luminal contents were not removed. Gross morphology images of pre-HS intestines were recorded immediately prior to replacement in the peritoneal cavity.

Following the intestinal preparations, the animals were heparinized to minimize clotting in catheters and in shed blood samples (1 U/mL of calculated blood volume i.v.; assuming 6 mL blood volume per 100 g total body weight) before onset of hemorrhage. MAP was reduced to 30 mmHg by withdrawal of blood through the venous catheter (0.4 mL/min) with a 5 mL syringe. The initial 1 mL of blood drawn was collected, centrifuged (1000 g, 5 min), and the plasma was immediately frozen at 80° C. for further analysis. The arterial pressure was monitored and adjusted by withdrawal/return of blood over the 90-min ischemic period. At the end of ischemia, the shed blood was returned to the animals (0.5 mL/min) plus 1 mL of saline (to replace the 1 mL of blood collected before shock). The animals remained anesthetized and were observed in the reperfusion phase for 3 h after initiation of the return of shed blood.

Gross morphology images of the intestine were captured at the end of the reperfusion period. Post-HS blood was collected through the arterial catheter, spun (1000 g, 5 min), and the plasma was immediately frozen at 80° C. The animals were then euthanized and lung and jejunum specimen were snap frozen for homogenization or embedded in optimum cutting temperature (O.C.T.) (Saukura, Leiden, the Netherlands) for histological sectioning. Jejunum was chosen for analysis, as more intestinal hemorrhage is typically seen there than in the ileum in models of HS.

As a positive control to demonstrate that active pancreatic proteases transport through the mesenteric lymph following shock, we collected mesenteric lymph and measured trypsin and chymotrypsin levels and activity over the course of intestinal ischemia. After a 3-cm midline laparotomy, the intestine was relocated from the abdomen and placed on a 1 cm platform to the left of the animal and covered with saline-soaked gauze and saran wrap to retain moisture, exposing the base of the mesentery and the superior mesenteric lymph vessel. A 1-cm bridge was placed under the animal to further expose the lymph vessel, which was then cleared of fat and surface fascia using blunt dissection. Silastic tubing (0.64 mm internal diameter) prefilled with heparin (10 mg/mL) to prevent clotting was drawn through the right abdominal wall using a suture needle and looped under the vena cava using curved tweezers. The superior mesenteric lymph vessel was cannulated with the tubing and held in place with VetBond tissue glue (3M, St. Paul, Minn.).

Once lymph fluid, visible as a white cloudy fluid, reached the other end of the cannula, this catheter end was placed inside a 2 mL tube preloaded with 5 μL of heparin. Fluid was collected for 1 h under the same conditions for every animal. Intestinal ischemia was induced by splanchnic arterial occlusion (SAO; N=5). Two microclamps were used to occlude the superior mesenteric artery and the celiac artery for the duration of the second hour, while animals in the Sham group (N=5) remained perfused. After the second hour, the clamps were removed from the SAO animals to begin reperfusion. Lymph fluid was collected continuously for another 3 h and aliquoted every hour. At the end of each hour, samples were centrifuged (1600 g, 4° C., 20 min) to remove cellular debris and excess fat, and the supernatant was stored at 80° C. After the 5 h of lymph fluid collection, animals were euthanized (120 mg/kg sodium pentobarbital i.v.).

After hemorrhagic shock, bronchoalveolar lavage fluid (BALF) was collected by inserting an 18 gauge blunt needle into the left main bronchus of the left lung. The lunch was photographed to assess gross morphological damage. The images were blindly assessed for macroscopic lung damage. Scoring was as follows: 0, no visible lung damage and pink in color; 1, mostly pink in color with some dark red lesions starting to form around the central part of the lung near the trachea; 2, some pink color with more lesions forming and spreading throughout the lung; 3, dark red lesions through the majority of the lung surface; 4, complete lung damage in all parts of the organ surface.

To collect BALF fluid, saline (˜2.0 ml/lavage) was injected and recovered three times before centrifugation (1000 g, 5 min). Following the third wash, the lung was injected with O.C.T., embedded, and frozen. The total volume of recovered BALF fluid was measured and the fraction of saline recovered versus the amount injected was used to normalize all measurements.

Intestinal Hemorrhage—Segments of jejunum were mechanically homogenized by weight (10 ml per 1 g; 18,000 g, 20 min; stored at −80° C.) in 0.5% hexadecyltrimethyl-ammonium bromide (HTAB) in PBS (pH 6.0). Any luminal content present in the segment was included in the weighing and homogenization. Erythrocyte infiltration, i.e. tissue hemorrhage, in each tissue homogenate was estimated by measuring absorbance at 405 nm, an absorbance peak for hemoglobin, on a plate reader. Intestinal bleeding was also assessed macroscopically by comparing photographs of the intestine before and after hemorrhagic shock.

Enzyme and Protease Activity Measurements—All microplate assays were performed in duplicate in 96 well black-sided flat bottom polystyrene plates (Corning, New York, N.Y.) and measured in a microplate reader (FilterMax F-5 Multi-mode, Molecular Devices, Sunnyvale, Calif.).

Erythrocyte infiltration, that is, hemorrhage, was estimated in homogenates of jejunum, including any luminal content present in the segment, by reading absorbance at 405 nm, an absorbance peak for hemoglobin, in duplicate on a plate reader. Intestinal bleeding was also assessed macroscopically by comparing photographs of the intestine before and after HS.

Protease activity was determined by digestion of the globular protein casein hybridized to Texas-red such that fluorescence intensity increases after cleavage (Enzchek protease assay kit; Life Technologies, Carlsbad, Calif.). Jejunal homogenates (10 μL) including any endogenous luminal contents present were mixed with casein substrate (90 μL) and loaded in duplicate. Plates were incubated in a prewarmed microplate reader and measurements were made every 5 min. The initial slope (i.e., change in caseinolytic activity per minute) was computed for each sample.

Myeloperoxidase (MPO) Activity Assay—In order to assess intestine and lung neutrophil infiltration, myeloperoxidase (MPO) activity was determined in the intestine and lung homogenates. Protein content was measured (BCA kit, ThermoScientific, Waltham, Mass.), then 40 μl of 2 mg/ml intestine homogenate or 20 μl of 1 mg/ml lung homogenate, were added to 180 μl of PBS (pH 6.0) mixed with 0.167 mg/ml o-dianisidine dihydrochloride (Sigma-Aldrich, St. Louis, Mo.) and 0.001% or 0.0005% H₂O₂ (w/v) for intestine and lung, respectively and measured as previously described.

To determine neutrophil MPO release into the BALF, 20 μl of BALF was loaded into each well together with 180 μl of PBS (pH 6.0) mixed with 0.167 mg/ml o-dianisidine dihydrochloride (Sigma-Aldrich) containing 0.001% H₂O₂. BALF protein concentration was also determined. All BALF calculations were normalized to the percent fluid recovered from three lavage collections.

Absorbance was measured kinetically at 450 nm every 5 min for 1 h at 37° C. As negative controls, 180 μL of PBS (pH 6.0) was added to 20 μL of sample, and the absorbance values of these samples were subtracted from the measurements with the substrate. The change in absorbance was linear within this period, and MPO activity is presented as the change in absorbance per minute per milligram of protein.

To measure the activity of specific pancreatic enzymes in lymph, a new electrophoretic method was used (Lefkowitz et al. 2010). The method is superior to colorimetric assays as the lymph often has an opacity (due to light diffraction) that interferes with absorbance measurements. These substrates are small peptide sequences that carry opposing charges at each end. When they are cleaved, the end with an attached fluorophore has a net positive charge, making it possible to isolate the cleaved segment with electrophoresis. A trypsin-specific substrate synthesized by Aapptec (Louisville, Ky.) was labeled on the lysine residue's e amine group with Bodipy FL-SE (Invitrogen, Carlsbad, Calif.). A similar chymotrypsin-specific peptide (Bodipy, FL) was also synthesized. Note that while the cleavage sites of these substrates are designed for trypsin and chymotrypsin, it is possible for other proteolytic enzymes with similar specificities to cleave them at a slower rate. Solutions of each substrate (0.5 mg/mL) were combined with 3.5 μL of lymph fluid in individual reaction tubes, and allowed to react for 1 h. Aliquots of each sample were electrophoresed at 500 volts (10 min), in a precast 20% polyacrylamide gel (Life Technologies). Fluorescent bands were quantified using a Storm 840 scanner (Molecular Dynamics, Sunnyvale, Calif.) configured to ImageQuant v5.2 software (General Electric, Fairfield, Conn.) using the following settings: fluorescence mode, high sensitivity, 100 mm pixel size, 1000 V photomultiplier tube with a 450 nm excitation filter and a 520 nm long pass emission filter. Digital images were integrated over each sample band followed by background subtraction (negative control, substrate+HCl) to obtain the final fluorescence values.

Protease Activity—Gelatin gel zymography was completed as previously described. When present, the luminal contents from the jejunal tissue were discarded by gentle compression of the intestine before homogenization to prevent excess digestion of the gelatin substrate in the gels by luminal proteases. Tissues from shocked animals (flushed and nonflushed) as well as controls were rinsed and blotted dry before weighing. Protein concentration was determined using the BCA kit before mixing each sample in a 1:1 ratio with loading dye (containing sodiumdodecyl sulfate (SDS), but no reducing agent). Two microgram protein/lane of intestine or 20 μg protein/lane of lung homogenates were loaded. Plasma (0.5 μL) was mixed with 2 μL loading dye and loaded into each lane.

Samples were separated by gel electrophoresis in 11% sodiumdodecyl sulfate polyacrylamide gel electrophoresis (SDS-PAGE) gelatin impregnated gels. Upon separation, gel proteins were renatured in 2.5% Triton-X 100 in water (49, 15 min) before incubation at 37° C. overnight in developing buffer (0.05 mol/L Tris base, 0.2 mol/L NaCl, 4 μmol/L ZnCl₂, 5 mmol/L CaCl₂.2H₂O). Gels were then fixed and stained (50% methanol, 10% acetic acid, 40% water, and 0.25% Coomassie blue solution) for 3 h, destained in water to achieve appropriate contrast before digital analysis (ImageJ).

To confirm that the ˜20-kDa bands were serine proteases, gels were renatured and developed in the aforementioned buffers with the addition of either 75 μmol/L serine protease inhibitor ANGD (nafamostat mesilate) or 100 lmol/L trypsin inhibitor tosyl-lysine chloromethyl ketone (TLCK) and compared to duplicate gels that were subject to the standard renaturation procedure.

Plasma MMP-1/9 activity was also detected by plate zymography using a protease specific substrate (American Peptide Company, Sunnyvale, Calif.).

Immunoblotting—Before homogenizing intestinal samples for immunoblotting, the luminal content was gently pressed out of the intestine for the No-HS and HS-NF samples. The intestines were rinsed in saline and blotted dry before weight determination. Lysis buffer (ThermoScientific) was mixed with a 1:100 dilution of 0.5 mol/L ethylenediaminetetraacetic acid (ThermoScientific) and a 1:50 dilution of HALT protease inhibitor cocktail. Lungs were homogenized in lysis buffer containing HALT protease inhibitor (1:100). A ratio of 1 mL of lysis buffer per 0.1 g of tissue was used for both organs.

For organ homogenates, protein concentration was calculated (BCA kit) so that 40 μg protein was loaded per well. Homogenates were mixed 1:1 with sample loading buffer (Bio-Rad, Hercules, Calif.) containing β-mercaptoethanol (0.05% by volume). Plasma was mixed in a 1:2 ratio of plasma:sample loading buffer containing b-mercaptoethanol (0.05% w/v), and 3 μL of this solution was loaded per well. Lymph fluid was mixed in a 1:1 ratio of lymph fluid:sample loading buffer containing β-mercaptoethanol (0.05% w/v), and 5 μL of this solution was loaded per well. Both tissue homogenates and plasma samples were denatured by boiling for 10 min prior to loading. Proteins were separated on SDS-PAGE gels (8% or 12% resolving, 4% stacking). Following separation, proteins were transferred onto a nitrocellulose membrane (Bio-Rad) and blocked for 1 h in 5% bovine serum albumin in trisbuffered saline with 0.5% Tween-20 (TBS-T). Membranes were cut longitudinally near the anticipated molecular weight of each protein and incubated overnight with the designated primary antibody (β-actin (sc-8432), trypsin (sc-137077), VE-cadherin (sc-6458), VEGF (sc-507), sc-VEGFR-2 (sc-48161) (Santa Cruz Biotechnologies, Santa Cruz, Calif.); chymotrypsin (ab-35694), MMP-9 (ab-76003) (Abcam); E-cadherin 33-4000 (Invitrogen).

Following incubation, the primary antibody was rinsed (TBS-T, 39, 10 min). Secondary antibodies against mouse, rabbit, or goat were diluted 1:10,000 in TBS-T and incubated with the membranes for 1 h. The membranes were washed (TBS-T, 39, 10 min) and developed using enhanced chemiluminescence substrate (ThermoScientific).

Membranes were scanned and bands digitally quantified (ImageJ). Lung and intestine bands were standardized by β-actin and normalized by the No-HS controls to determine fold changes in protein levels. Plasma protein levels were not normalized.

Immunohistochemistry—Mucin distribution was assessed using immunohistochemistry after HS. Sections were allowed to dry for 20 min before fixation in ice-cold acetone at 20° C. for 10 min. Following fixation, slides were washed in PBS (29, 3 min). Endogenous hydrogen peroxidase activity was quenched (95% methanol, 3% hydrogen peroxide for 5 min). Slides were washed again in PBS (29, 3 min). Individual sections were isolated with a hydrophobic pen (Vector Labs, Burlingame, Calif.) before blocking with 2.5% normal horse serum (Vector Labs) for 30 min. A primary antibody against mucin 13 (Santa Cruz Biotechnology; sc-66973) was diluted 1:200 in 1.25% normal horse serum and incubated with the sections overnight at 4° C. The primary antibody was washed with PBS (39, 5 min) and sections were incubated with anti-rabbit secondary reagent (Immpress Kit; Vector Labs) for 30 min. Secondary antibody was removed by washing with PBS (39, 5 min). Colorimetric labeling was achieved by using DAB (3,3′-diaminobenzidine) substrate (Vector Labs) for 30 sec followed by rinsing in water. Sections were finally counterstained with Toluidine blue (0.05% in 1% boric acid) for 5 sec. Slides were washed with three changes of deionized (DI) water before dehydrating in an ethanol gradient (70%, 95%, and 100%) and cleaning with xylene. Slides were allowed to dry overnight before mounting with hard set mounting media (Vector Labs).

Lung histology—Lung sections were allowed to dry for 20 min before fixation in ice-cold methanol at 20° C. Slides were washed in DI water (Bounous 1967) and incubated with hematoxylin solution (Vector Labs) for 30 sec. They were rinsed thoroughly in DI water and incubated in eosin (Fisher Scientific, Waltham, Mass.) for 30 sec followed by additional rinses in DI water. The slides were dehydrated in 70%, 95%, and 100% ethanol before cleaning with xylene and were dried overnight before being mounted with hard set mounting media (Vector Labs).

Statistical Analysis—Results are presented as mean±SD. Paired t-tests were completed for comparisons between groups between pre- and post-shock plasma samples. Mann-Whitney tests were used for non-normally distributed data. ANOVA followed by Tukey post-hoc analysis was used for normally distributed parametric variables. Statistical analysis was performed with commercial software (OriginLabs, Northampton, Mass.).

Results—Intestinal Injury and Protease Activity—The blood pressure between the HS-NF and HS-F animals did not significantly differ over the observation period of 180 min. As expected, given the presence of luminal content and proteases, the intestinal proteolytic activity as determined by casein substrate digestion in the HS-NF animals was significantly higher compared to flushed animals. Additionally, the activity increased in the HS-NF group compared to the No-HS group, suggesting local activation and/or influx from the circulation of proteases. MMP-9, detected by immunoblot, was elevated in the HS-NF animals compared to the No-HS animals. All animals had intestines without lesions prior to the onset of ischemia/reperfusion injury, but the intestines developed microhemorrhages along the length of the intestine in both the jejunum and ileum regions of the HS-NF group only. These lesions appeared more intense at sites where food was present. In contrast, none of the six HS-F animals had macroscopic lesions except near the surgical sites at the proximal jejunum and distal ileum (a feature shared by the HS-F and HS-NF animals). Though the intestinal homogenate absorbance (at 405 nm) as a quantitative marker for hemoglobin escape was not significantly different between the groups (P=0.08 between No-HS and HS-NF), the hemoglobin absorbance linearly correlated with the degree of caseinolytic activity in the nonflushed samples (R²=0.88; not shown).

The MAP after hemorrhagic shock/reperfusion did not differ among groups treated with a single intervention at the end of the reperfusion period (FIG. 7A). Macroscopic assessment of the intestine after shock depicted microhemorrhages most severely in HS+SAL and HS+GLUC groups while the HS+DOX and HS+TA groups had fewer regions with microhemorrhages (FIG. 7B). Hemoglobin absorbance of whole intestinal homogenates also followed this trend, though there were no statistically significant differences between groups (FIG. 7C). MPO activity of intestinal homogenates tended to be lower with TA or glucose treatments, whereas doxycycline increased MPO activity (FIG. 7D).

Although macroscopic damage to the intestine differed between groups, all the post-HS samples had elevated serine protease activity (FIG. 8A), and these disappeared if the gels were renatured and developed with serine protease specific inhibitors (nafamostat mesilate and tosyllysine chloromethyl ketone hydrochloride). However, levels of trypsin and chymotrypsin in the plasma were unchanged after HS (FIG. 8B).

Pre- and post-plasma samples were tested for MMP-1/9 activity using a specific substrate. MMP-1/9 activity was reduced by 85% in the HS+DOX animals compared to the HS+SAL animals. MMP-9 and neutrophil derived pro-MMP-9 were elevated after HS in all groups, and HS+GLUC treated animals had significantly more activity compared to HS+SAL (FIG. 8C). Although MMP-9 activity and levels increased after HS, TIMP-1 levels were unchanged at this time point (FIG. 8D).

Histological sections of the villi revealed shedding of epithelial bound mucin 13 into the lumen of the intestine after HS in both groups, though more pronounced in the HS-F group due to the lack of preexisting luminal contents. Although the distribution of mucin 13 changed after HS, the levels as determined by immunoblot did not. The E-cadherin bands of the intestinal epithelial cells were reduced in the HS-NF animals only, and the tight junction protein occludin band did not change.

Results—Transport of serine proteases from the gut—Mesenteric lymph fluid was collected to study pancreatic enzyme activity and transport from the gut in animals with innate luminal contents after intestinal ischemia. Before intestinal ischemia, trypsin and chymotrypsin activities were detected in the nanomolar range, but after that the activities doubled and remained elevated throughout the reperfusion period. To confirm the presence of pancreatic trypsin and chymotrypsin in the lymph, monoclonal antibodies against rat pancreatic trypsin or chymotrypsin were used to successfully detect these proteins in the lymph fluid, although their levels did not change after intestinal ischemia.

Likewise, both trypsin and chymotrypsin were detected at equal levels in pre- and post-HS plasma in HS; flushing the intestine did not affect their levels. Low molecular-weight bands (˜20 kDa) were present in gelatin gel zymography in post-HS plasma samples only in the HS-F and the HS-NF groups. As the lung is the first organ that mesenteric lymph fluid encounters upon mixing with venous return blood, a check for low molecular weight band formation was performed in the lung homogenates and it was found that elevated activity existed in the HS-NF animals only. To confirm that these bands were serine proteases, gels were renatured with a broad spectrum serine protease inhibitor ANGD or trypsin-specific inhibitor (TLCK) and found a substantial decrease in the serine protease band intensities in the gels from plasma, lung, and intestine samples.

Results—MMP-9 activity and levels after HS—Previous studies have shown that MMP-9 is elevated in the plasma and lungs after HS (Altshuler et al. 2012), and it was investigated whether the activation differed if luminal contents were present as active trypsin in the systemic circulation can directly convert pro-MMPs into their active forms (Duncan et al. 1998). The MMP-9 total protein levels (samples reduced by β-mercaptoethanol) in the plasma increased in both HS-F and HS-NF animals, but the HS-F animals were significantly lower compared to the HS-NF animals. After HS, the protease activity of both active MMP-9 and pro-MMP-9 (the pro-forms of MMP are activated by the denaturation/renaturation in gel zymography; 125 kDa derived from neutrophils (Olson et al. 2000)) increased after shock in the plasma in the HS-F and HS-NF animals. The MMP-9 dimers also increased after HS in the plasma, but there was no difference between the HSNF and HS-F groups. There were no changes in the density of MMP-2 or pro-MMP-2. The MMP-9 levels in the lung were elevated after HS in the lung homogenate as detected by immunoblot. Neutrophil-derived pro-MMP-9 (125 kDa) activity determined by gel zymography was elevated in both groups, and MMP-9 activity was also elevated but the HS-NF was significantly elevated compared to the HS-F group, possibly reflecting activation by active serine proteases in the lung. There were no changes in the levels of MMP-9 dimers that formed. It was confirmed that these molecular weights are equivalent to the activity bands derived from neutrophils. There were no changes in MMP-9 or MMP-2 activity in the lymph fluid as determined by gelatin gel zymography between SAO and Sham animals throughout each time course.

Results—Lung Injury—Regardless of the individual treatment, the MPO activity in lung homogenates was elevated after HS (FIG. 9A). The average MPO activity and the protein concentration in the BALF were elevated after HS, but not statistically significant (FIG. 9A). Macroscopic inspection of lungs showed an increase in injury in the HS+SAL animals compared to No-HS and a trend in reduction with glucose treatment. Serine protease activity bands were increased in the lung homogenates with HS and pancreatic trypsin was detected in all lung homogenates (FIG. 9B). MMP-9 activity and levels were elevated in lung homogenates, but to a lesser extent in the HS+DOX animals (FIG. 9C).

Lung junctional proteins occludin, VE-cadherin, and E-cadherin were degraded after HS in all groups (FIG. 10A). The endothelial survival protein VEGFR-2 also decreased after HS, but was maintained in the HS+TA group (FIG. 10B), as compared to the NO-HS group without shock. The plasma levels of the 38 kDa form of VEGF were significantly elevated after HS in all groups (FIG. 10C).

Results—Multiple Treatments—Gross morphology of the intestine was improved in the animals given DOX+TA, TA+GLUC, and DOX+TA+GLUC but microhemorrhages continued to form in the HS+DOX+GLUC animals (FIG. 11A). The mean arterial pressure decreased at a significantly slower rate in the HS+DOX+TA+GLUC group compared to the HS+SAL group (FIG. 11B). Multiple interventions did not reduce the hemorrhage into the intestinal lumen, the lung injury score, the MPO activity in the intestine and lung, or the BALF protein levels. Serine protease and MMP-9 activities in the plasma and lung remained elevated in all combination treatments. However, the lung VE- and the intestinal E-cadherin levels were not significantly degraded compared to the untreated HS animals (FIG. 11C), but treatments were unable to significantly preserve VEGFR-2.

Although the invention has been described with reference to the above example, it will be understood that modifications and variations are encompassed within the spirit and scope of the invention. All references cited herein are incorporated herein by reference in their entireties. Accordingly, the invention is limited only by the following claims.

What is claimed is:

1. A method for preventing breakdown of a mucosal barrier in the intestine of a subject in need thereof comprising administering to the lumen of the intestine an effective amount of glucose, thereby preventing breakdown of the mucosal barrier in the intestine.

2. The method of claim 1, wherein the glucose is administered to the subject orally, by nasogastric intubation (NG tube), by catheter into the duodenum or upper jejunum, or by direct administration to the small intestine.

3. The method of claim 1, further comprising administering an effective dose of a matrix-degrading metalloproteinase (MMP) inhibitor to the subject.

4. The method of claim 1, wherein the MMP inhibitor is selected from the group consisting of doxycycline, minocycline, minocycline analogs, tetracyclin-based inhibitors, hydroxamate-, thiol-, phosphorus-, pyrimidine-based inhibitors, iliomastat, tranexamic acid, endogenous tissue inhibitors of metalloproteinase (TIMPs), grape seed extract, resveratrol, and GM-6001.

5. The method of claim 1, further comprising administering an effective dose of a serine protease inhibitor to the subject.

6. The method of claim 5, wherein the serine protease inhibitor is selected from the group consisting of tranexamic acid, 6-amidino-2-naphthyl p-guanidinobenzoate dimethanesulfonate nafamostat mesilate (ANGD), gabaxate mesilate (Foy), trasylol, alpha 1-antitrypsin, kallikrein, neutrophil elastase inhibitor, plasminogen activator inhibitor-1, and alpha 1-antichymotrypsin (α₁AC).

7. A method for treating sepsis in a subject in need thereof comprising administering to the lumen of the intestine of the subject an effective amount of glucose.

8. The method of claim 7, wherein the subject suffers from intestinal complications associated with hypoxia, trauma, hypothermia, burn, systemic infections, intestinal infections, inflammatory bowel conditions, anesthesia, radiation and/or chemotherapy, drug/alcohol overdose, or any condition associated with intestinal underperfusion.

9. The method of claim 8, wherein the condition associated with intestinal underperfusion is selected from the group consisting of elevated central venous blood pressure, mesentery artery occlusion, and aortic occlusion.

10. The method of claim 7, wherein the glucose is administered to the subject orally, by nasogastric intubation (NG tube), by catheter into the duodenum or jejunum, or by direct administration to the small intestine.

11. The method of claim 7, further comprising administering an effective dose of a matrix-degrading metalloproteinase (MMP) inhibitor to the subject.

12. The method of claim 7, wherein the MMP inhibitor is selected from the group consisting of doxycycline, minocycline, minocycline analogs, tetracyclin-based inhibitors, hydroxamate-, thiol-, phosphorus-, pyrimidine-based inhibitors, iliomastat, tranexamic acid, endogenous tissue inhibitors of metalloproteinase (TIMPs), grape seed extract, resveratrol, and GM-6001.

13. The method of claim 7, further comprising administering an effective dose of a serine protease inhibitor to the subject.

14. The method of claim 13, wherein the serine protease inhibitor is t tranexamic acid, 6-amidino-2-naphthyl p-guanidinobenzoate dimethanesulfonate nafamostat mesilate (ANGD), gabaxate mesilate (Foy), trasylol, alpha 1-antitrypsin, kallikrein, neutrophil elastase inhibitor, plasminogen activator inhibitor-1, and alpha 1-antichymotrypsin (α₁AC).

15. A method of preventing intestinal ischemia in a trauma patient comprising administering an effective dose of glucose into the lumen of the intestine of the subject and an effective dose of a MMP inhibitor.

16. The method of claim 15, wherein the subject suffers from intestinal complications associated with hypoxia, trauma, hypothermia, burn, inflammatory bowel conditions, systemic and intestinal infection, anesthesia, radiation, chemotherapy, drug/alcohol overdose, or any condition associated with intestinal underperfusion.

17. The method of claim 16, wherein the condition associated with intestinal underperfusion is selected from the group consisting of elevated central venous blood pressure, mesentery artery occlusion, and aortic occlusion.

18. The method of claim 15, wherein the glucose is administered to the subject orally, by nasogastric intubation (NG tube), by catheter into the duodenum or jejunum, or by direct administration to the small intestine.

19. The method of claim 15, wherein the MMP inhibitor is selected from the group consisting of doxycycline, minocycline, minocycline analogs, tetracyclin-based inhibitors, hydroxamate-, thiol-, phosphorus-, pyrimidine-based inhibitors, iliomastat, tranexamic acid, endogenous tissue inhibitors of metalloproteinase (TIMPs), grape seed extract, resveratrol, and GM-6001.

20. The method of claim 15, further comprising administering an effective dose of a serine protease inhibitor to the subject.

21. The method of claim 20, wherein the serine protease inhibitor is selected from the group consisting of tranexamic acid, 6-amidino-2-naphthyl p-guanidinobenzoate dimethanesulfonate nafamostat mesilate (ANGD), gabaxate mesilate (Foy), trasylol, alpha 1-antitrypsin, kallikrein, neutrophil elastase inhibitor, plasminogen activator inhibitor-1 and alpha 1-antichymotrypsin (α₁AC).

22. A method of preventing or treating intestinal ischemia in a trauma patient comprising administering an effective dose of glucose into the lumen of the intestine of the subject and an effective dose of a serine protease inhibitor, thereby minimizing opening of the mucosal barrier to digestive enzymes.

23. The method of claim 22, wherein the subject suffers from intestinal complications associated with hypoxia, trauma, hypothermia, burn, inflammatory bowel conditions, systemic and intestinal infection, anesthesia, radiation and/or chemotherapy, drug/alcohol overdose, or any condition associated with intestinal underperfusion.

24. The method of claim 23, wherein the condition associated with intestinal underperfusion is selected from the group consisting of elevated central venous blood pressure, mesentery artery occlusion, and aortic occlusion.

25. The method of claim 22, wherein the glucose is administered to the subject orally, by nasogastric intubation (NG tube), by catheter into the duodenum, or by direct administration to the small intestine.

26. The method of claim 22, wherein the serine protease inhibitor is selected from the group consisting of tranexamic acid, 6-amidino-2-naphthyl p-guanidinobenzoate dimethanesulfonate nafamostat mesilate (ANGD), gabaxate mesilate (Foy), trasylol, alpha 1-antitrypsin, kallikrein, neutrophil elastase inhibitor, plasminogen activator inhibitor-1, and alpha 1-antichymotrypsin (α₁AC).

27. The method of claim 22, further comprising administering an effective dose of a MMP inhibitor to the subject.

28. The method of claim 27, wherein the MMP inhibitor is selected from the group consisting of doxycycline, minocycline, minocycline analogs, tetracyclin-based inhibitors, hydroxamate-, thiol-, phosphorus-, pyrimidine-based inhibitors, iliomastat, tranexamic acid, endogenous tissue inhibitors of metalloproteinase (TIMPs), grape seed extract, resveratrol, and GM-6001.
